SEQUENCE LISTING (226 sequences)

SEQ ID NO: 1 - DNA, 650 bp, Homo sapiens
  CDS (91)...(588)
caggaattctgtttggaaactgtcatgaaatacctgctaccgaccgcggctgcaggcctgctcctgcttgccgcgcaacccgccatggccatggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctgaactctcataccatggaagcttgcagtagcgagtagcatttttttcatggtgttattcccgat

SEQ ID NO: 2 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 3 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaatattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggtggtttctggcctgcgcagccttaccacgctgctccgtgcactgggtgcccaaaaagaagctatctcgtcgcctgacgcggcctcagcagcgccgttacgcactattacagccgataccttccgtaaactgtttcgcgtctacgccaacttcttgcgtggcaaactgaaactttatacgggtgaggcttgtcgctga

SEQ ID NO: 4 - protein, 165 aa, Homo sapiens
MDIAPPRLICDSRVLERYLLEAKEAENITTGCAEYCSLNENITVPDTKVNFYAWKRMEVGQQAVEVWQGLALLSEAVLRGQTLLVNSSQPWEPLQLHVDKVVSGLRSLTTLLRALGAQKEAISSPDAASAAPLRTITADTFRKLFRVYANFLRGKLKLYTGEACR

SEQ ID NO: 5 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgtagaagattgctcactgaacgagaatattactgtaccggatacgaaagtcaacttttatgcccggaaacgaatggaagttggacaacaggcggtggaaatttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 6 - protein, 165 aa, Homo sapiens
MDIAPPRLICDSRVLERYLLEAKEAETITTGCVEDCSLNENITVPDTKVNFYARKRMEVGQQAVEIWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 7 - DNA, 498 bp, Homo sapiens
  CDS (1)...(498)
atggatatcgccccgccccgtctgatttgcgacagcagggtgctagaaagatacctgctcgaagcgaaagaggctgaaagtatcactacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagtcttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaagtttatacgggtgaggcttgtcgctga

SEQ ID NO: 8 - protein, 165 aa, Homo sapiens
MDIAPPRLICDSRVLERYLLEAKEAESITTGCAEHCSLNENITVPDTKVNFYAWKRMEVGQQAVEVWQGLALLSEAVLRGQTLLVSSSQSWEPLQLHVDKAVSGLRSLTTLLRALGAQKEAISPPDAASAAPLRTITADTFRKLFRVYSNFLRGKLKVYTGEACR

SEQ ID NO: 9 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacatttatgcctggaaacgaatggaagttggacaacaggcggtggaagtttggcaggggcttgccctgttgtcggaggcagtcctgcggggtcaaactttactggtaatttccagtcagccttgggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 10 - protein, 165 aa, Homo sapiens
MDIAPPRLICDSRVLERYLLEAKEAENITTGCAEHCSLNENITVPDTKVNIYAWKRMEVGQQAVEVWQGLALLSEAVLRGQTLLVISSQPWELLQLHVDKAVSGLRSLTTLLRALGAQKEAISPPDAASAAPLRTITADTFRKLFRVYSNFLRGKLKLYTGEACR

SEQ ID NO: 11 - DNA, 498 bp, Homo sapiens
  CDS (1)...(498)
atggatatcaccccgccccgtctgatttgcgacagcagggtgctagaaagatacctgctcgaagcgaaagaggctgaaaatatcaccacaggctgtgtagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 12 - protein, 165 aa, Homo sapiens
MDITPPRLICDSRVLERYLLEAKEAENITTGCVEHCSLNENITVPDTKVNFYAWKRMEVGQQAVEVWQGLALLSEAVLRGQTLLVISSQPWELLQLHVDKAVSGLRSLTTLLRALGAQKEAISPPDAASAAPLRTITADTFRKLFRVYSNFLRGKLKLYTGEACR

SEQ ID NO: 13 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcttggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 14 - protein, 165 aa, Homo sapiens; identical to SEQ ID NO: 2

SEQ ID NO: 15 - DNA, 498 bp, Homo sapiens
  CDS (1)...(498)
atggatatcgccccgccccgtctgatttgcgacagcagggcgctagaaagatacctgctcgaagcgaaagaggctgaaattatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagcctagggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaactttatacgggtgaggcttgtcgctga

SEQ ID NO: 16 - protein, 165 aa, Homo sapiens
MDIAPPRLICDSRALERYLLEAKEAEIITTGCAEHCSLNENITVPDTKVNFYAWKRMEVGQQAVEVWQGLALLSEAVLRGQTLLVNSSQPREQLQLHVDKAVSGLRSLTTLLRALGAQKEAISPPDAASAAPLRTITADTFRKLFRVYPNFLRGKLKLYTGEACR

SEQ ID NO: 17 - DNA, 498 bp, Homo sapiens
  CDS (1)...(498)
atggatatcgccccgccccgtctgatttgcgacagcagggtgctagaaagatacctgttcgaagcgaaagaggctgaaaatatcaccacaggctgtgcagaacattgctcactgaacgagattattactgtaccggatccgaaagtcaacctttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 18 - protein, 165 aa, Homo sapiens
MDIAPPRLICDSRVLERYLFEAKEAENITTGCAEHCSLNEIITVPDPKVNLYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 19 - DNA, 498 bp, Homo sapiens
  CDS (1)...(498)
atggatatggcccggcccggtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaagggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaacttaaactttatacgggtgaggcttgtcgctga

SEQ ID NO: 20 - protein, 165 aa, Homo sapiens
MDMARPGLICDSRVLERYLLEAKEAENITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 21 - DNA, 499 bp, Homo sapiens
  CDS (2)...(499)
catggatatcgccccgccccgtctgatttgcgacagcagggtgctagaaagatacctgctcgaagcgaaagaggctgaaaatatcaccacaggctgtgaagaatattgctcactgaacgagattattactgtaccggattcgaaagtcaacttgtatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 22 - protein, 165 aa, Homo sapiens
MDIAPPRLICDSRVLERYLLEAKEAENITTGCEEYCSLNEIITVPDSKVNLYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 23 - DNA, 499 bp, Homo sapiens
  CDS (2)...(499)
catggatatcgccccgccccgtctgatttgcgacagcagggtgctagaaagatacctgctcgaagcgaaagaggctgaaaatatcaccacaggctgtgcagaacattgctcactgaacgagaatattactgtaccggatccgaaagtcaacttgtatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 24 - protein, 165 aa, Homo sapiens
MDIAPPRLICDSRVLERYLLEAKEAENITTGCAEHCSLNENITVPDPKVNLYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 25 - DNA, 499 bp, Homo sapiens
  CDS (2)...(499)
catggatatcgccccgccccgtctgatttgcgacagcagggtgctagaaagatacctgctcgaagcgaaagaggctgaaattatcaccacaggctgtgcagaacattgcttactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 26 - protein, 165 aa, Homo sapiens
MDIAPPRLICDSRVLERYLLEAKEAEIITTGCAEHCLLNENITVPDTKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKFYTGEACR

SEQ ID NO: 27 - DNA, 498 bp, Homo sapiens
  CDS (1)...(498)
atggatatcgtcccgccccgtctgatttgcgacagcagggtgctagaaagatacctgctcgaagcgaaagaggctgaaaatatcaccacaggctgtgtagaagattgctcactgaacgagaatattactgtaccggatccgaaagtcaacttttatgcctggaaacgaatggaagttggacaacaggcggtggaagtttggcaggggcttgccctgttgtcggaggcagtcctgcggggtcaaactttactggtaaattccagtcagtctggggaacgattacagttgcacgtggataaggcggtttctggactgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaactttatacgggtgaggcttgtcgctga

SEQ ID NO: 28 - protein, 165 aa, Homo sapiens
MDIVPPRLICDSRVLERYLLEAKEAENITTGCVEDCSLNENITVPDPKVNFYAWKRMEVGQQAVEVWQGLALLSEAVLRGQTLLVNSSQSGERLQLHVDKAVSGLRSLTTLLRALGAQKEAISPPDAASAAPLRTITADTFRKLFRVYANFLRGKLKLYTGEACR

SEQ ID NO: 29 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggtttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 30 - protein, 165 aa, Homo sapiens
MDIAPPRLICDSRVLERYLLEAKEAENITTGCAEHCSLNENITVPDTKVNFYAWKRMEVGQQAVEVWQGFALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 31 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtagcaaactgaaactttatacgggtgaggcttgtcgctga

SEQ ID NO: 32 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRSKLKLYTGEACR

SEQ ID NO: 33 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaatgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcaacagcgccgttacgcactattacagccgataccttccgtaaactgtttcgcgtctactccaacttcttgcgtggcaaactgaaactttatacgggtgaggcttgtcgctga

SEQ ID NO: 34 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASTAPLRTITADTFRKLFRVYSNFLRGKLKLYTGEACR

SEQ ID NO: 35 - DNA, 498 bp, Homo sapiens
  CDS (1)...(498)
atggatattgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtgaaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggtttgtcgctga

SEQ ID NO: 36 - protein, 165 aa, Homo sapiens
MDIAPPRLICDSRVLERYLLEAKEAENITTGCAEHCSLNENITVPDTKVNFYAWKRMEVGQQAVKVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEVCR

SEQ ID NO: 37 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggattcgaaagtcaacttatatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 38 - protein, 165 aa, Homo sapiens
MDIAPPRLICDSRVLERYLLEAKEAENITTGCAEHCSLNENITVPDSKVNLYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 39 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagattattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaagtttatacgggtgaggcttgtcgctga

SEQ ID NO: 40 - protein, 165 aa, Homo sapiens
MDIAPPRLICDSRVLERYLLEAKEAENITTGCAEHCSLNEIITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYANFLRGKLKVYTGEACR

SEQ ID NO: 41 - DNA, 498 bp, Homo sapiens
  CDS (1)...(498)
atggatatcgccccgtcccgtctgatttgcgacagcagggtgctagaaagatacctgctcgaagcgaaagaggctgaaactatcaccacaggctgtggagaaaattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagcctggggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 42 - protein, 165 aa, Homo sapiens
MDIAPSRLICDSRVLERYLLEAKEAETITTGCGENCSLNENITVPDTKVNFYAWKRMEVGQQAVEVWQGLALLSEAVLRGQTLLVNSSQPGELLQLHVDKAVSGLRSLTTLLRALGAQKEAISPPDAASAAPLRTITADTFRKLFRVYSNFLRGKLKLYTGEACR

SEQ ID NO: 43 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgaagaatattgctcactgaacgagaatattactgtaccggatacgaaagtcaacttttatgcccggaaacgaatggaagttggacaacaggcggtggaagtttggcaggggcttgccctgttgtcggaggcagtcctgcggggtcaaactttactggtaaattccagtcaggctcgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaagtttatacgggtgaggcttgtcgctga

SEQ ID NO: 44 - protein, 165 aa, Homo sapiens
MDIAPPRLICDSRVLERYLLEAKEAESITTGCEEYCSLNENITVPDTKVNFYARKRMEVGQQAVEVWQGLALLSEAVLRGQTLLVNSSQAREPLQLHVDKAVSGLRSLTTLLRALGAQKEAISPPDAASAAPLRTITADTFRKLFRVYSNFLRGKLKVYTGEACR

SEQ ID NO: 45 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatgcgaaagtcaacttatatgcctggaaacgaatggaagttggacaacaggcggtggaagtttggcaggggcttgccctgttgtcggaggcagtcctgcggggtcaaactttactggtaaattccagtcagactggggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaagtttatacgggtgaggcttgtcgctga

SEQ ID NO: 46 - protein, 165 aa, Homo sapiens
MDIAPPRLICDSRVLERYLLEAKEAENITTGCAEHCSLNENITVPDAKVNLYAWKRMEVGQQAVEVWQGLALLSEAVLRGQTLLVNSSQTGEQLQLHVDKAVSGLRSLTTLLRALGAQKEAISPPDAASAAPLRTITADTFRKLFRVYSNFLRGKLKVYTGEACR

SEQ ID NO: 47 - DNA, 498 bp, Homo sapiens
  CDS (1)...(498)
atggatatcgccccgccccgtctgatttgcgacagcagggtgctagaaagatacctgcttgaagcgaaagaggctgaaagtatcaccacaggctgtgcagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagtctcgggaacgattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 48 - protein, 165 aa, Homo sapiens
MDIAPPRLICDSRVLERYLLEAKEAESITTGCAEDCSLNENITVPDTKVNFYAWKRMEVGQQAVEVWQGLALLSEAVLRGQTLLVNSSQSRERLQLHVDKAVSGLRSLTTLLRALGAQKEAISPPDAASAAPLRTITADTFRKLFRVYSNFLRGKLKLYTGEACR

SEQ ID NO: 49 - DNA, 503 bp, Homo sapiens
  CDS (1)...(498)
atggatatcgccccgccccgtctgatttgcgacagcagggtgctagaaagatacctgctcgaagcgaaagaggctgaaactatcaccacaggctgtgtagaatattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttttggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctgaactct

SEQ ID NO: 50 - protein, 165 aa, Homo sapiens
MDIAPPRLICDSRVLERYLLEAKEAETITTGCVEYCSLNENITVPDTKVNFYAWKRMEVGQQAVEVWQGLALLSEAVLRGQTLLVNSSQPWEPLQLHVDKAVFGLRSLTTLLRALGAQKEAISPPDAASAAPLRTITADTFRKLFRVYSNFLRGKLKLYTGEACR

SEQ ID NO: 51 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatgcgaaagtcaacttttatgcccggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaagtttatacgggtgaggcttgtcgctga

SEQ ID NO: 52 - protein, 165 aa, Homo sapiens
MDIAPPRLICDSRVLERYLLEAKEAENITTGCAEHCSLNENITVPDAKVNFYARKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKVYTGEACR

SEQ ID NO: 53 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataagacggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaaatttatacgggtgaggcttgtcgctga

SEQ ID NO: 54 - protein, 165 aa, Homo sapiens
MDIAPPRLICDSRVLERYLLEAKEAENITTGCVEDCSLNENITVPDTKVNFYAWKRMEVGQQAVEVWQGLALLSEAVLRGQTLLVISSQPWEPLQLHVDKTVSGLRSLTTLLRALGAQKEAISPPDAASAAPLRTITADTFRKLFRVYSNFLRGKLKIYTGEACR

SEQ ID NO: 55 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagccttgggaaccattacagttgcacgtggataaggcggtttttggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 56 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVSSSQPWEPLQLHVDKAVFGLRSLTTLLRALGAQKEAISPPDAASAAPLRTITADTFRKLFRVYSNFLRGKLKLYTGEACR

SEQ ID NO: 57 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacggaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 58 - protein, 165 aa, Homo sapiens
MDIAPPRLICDSRVLERYLLEAKEAENITTGCAEHCSLNENITVPDTEVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKFYTGEACR

SEQ ID NO: 59 - DNA, 498 bp, Homo sapiens
  CDS (1)...(498)
atggatatcgccccgccccgtctgatttgcgacagcagggtgttagaaagatacctgctcgaagcgaaagaggctgaaaatatcaccacaggctgtgcagaacattgctcactgaacgagattattactgtaccggatccgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaaatttatacgggtgaggcttgtcgctga

SEQ ID NO: 60 - protein, 165 aa, Homo sapiens
MDIAPPRLICDSRVLERYLLEAKEAENITTGCAEHCSLNEIITVPDPKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKIYTGEACR

SEQ ID NO: 61 - DNA, 498 bp, Homo sapiens
  CDS (1)...(498)
atggatatcgccccgccccgtctgatttgcgacagcagggtgctagaaagatacctgctcgaagcgaaagaggctgaaaatatcatcacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagacttgggaacaattacagttgcacgtggataaggcggtttctggcctgcgcagccttaccacgctgctccgtgcactgggtgcccaaaaagaagctatctcgccgcctgacgcggcctcagcagcgctgttacgcactattacagccgataccttccgtaaactgtttcgcgtctacaccaacttcttgcgtggcaaactgaaagtttatacgggtgaggcttgtcgctga

SEQ ID NO: 62 - protein, 165 aa, Homo sapiens
MDIAPPRLICDSRVLERYLLEAKEAENIITGCAEHCSLNENITVPDTKVNFYAWKRMEVGQQAVEVWQGLALLSEAVLRGQTLLVNSSQTWEQLQLHVDKAVSGLRSLTTLLRALGAQKEAISPPDAASAALLRTITADTFRKLFRVYTNFLRGKLKVYTGEACR

SEQ ID NO: 63 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcagggacttgccctgttgtcggaggcagtcctgcggggtcaaactttactggtaacttccagtcaggctcgggaacgattacagttgcacgtggataaggcggtttctggcctgcgcagccttaccacgctgctccgtgcactgggtgcccaaaaagaagctatctcgccgcctgacgcggcctcagcagcgccgttacgcactattacggccgataccttccgtaaactgtttcgcgtctactccaacttcttgcgtggcaaactgaaactttatacgggtgaggcttgtcgctga

SEQ ID NO: 64 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVTSSQARERLQLHVDKAVSGLRSLTTLLRALGAQKEAISPPDAASAAPLRTITADTFRKLFRVYSNFLRGKLKLYTGEACR

SEQ ID NO: 65 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtggagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaagtttatacgggtgaggcttgtcgctga

SEQ ID NO: 66 - protein, 165 aa, Homo sapiens
MDIAPPRLICDSRVLERYLLEAKEAENITTGCGEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYANFLRGKLKVYTGEACR

SEQ ID NO: 67 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaacattgctcactgaacgagagtattactgtaccggatgcgaaagtcaacttttatgcctggaaacgaatggaagttggacaacaggcggtggaagtttggcaggggcttgccctgttgtcggaggcagtcctgcggggtcaaactttactggtaaattccagtcaggcttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaactttatacgggtgaggcttgtccctga

SEQ ID NO: 68 - protein, 165 aa, Homo sapiens
MDIAPPRLICDSRVLERYLLEAKEAEIITTGCAEHCSLNESITVPDAKVNFYAWKRMEVGQQAVEVWQGLALLSEAVLRGQTLLVNSSQAWEPLQLHVDKAVSGLRSLTTLLRALGAQKEAISPPDAASAAPLRTITADTFRKLFRVYTNFLRGKLKLYTGEACP

SEQ ID NO: 69 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacgtctatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaaatttatacgggtgaggcttgtcgctga

SEQ ID NO: 70 - protein, 165 aa, Homo sapiens
MDIAPPRLICDSRVLERYLLEAKEAENITTGCAEHCSLNENITVPDTKVNVYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYANFLRGKLKIYTGEACR

SEQ ID NO: 71 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgtactattacagccgataccttccgtaaactgtttcgcgtctactccaacttcttgcgtggcaaactgaaaatttatacgggtgaggcttgtcgctga

SEQ ID NO: 72 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVSSSQPWEPLQLHVDKAVSGLRSLTTLLRALGAQKEAISPPDAASAAPLRTITADTFRKLFRVYSNFLRGKLKIYTGEACR

SEQ ID NO: 73 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgaagaaaattgctcactgaacgagagtattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagtctcgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaattttatacgggtgaggcttgtcgctga

SEQ ID NO: 74 - protein, 165 aa, Homo sapiens
MDIAPPRLICDSRVLERYLLEAKEAENITTGCEENCSLNESITVPDTKVNFYAWKRMEVGQQAVEVWQGLALLSEAVLRGQTLLVISSQSREPLQLHVDKAVSGLRSLTTLLRALGAQKEAISPPDAASAAPLRTITADTFRKLFRVYANFLRGKLKFYTGEACR

SEQ ID NO: 75 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgttcactgaacgagaatattactgtaccggattcgaaagtcaacttgtatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaactttatacgggtgaggcttgtcgctga

SEQ ID NO: 76 - protein, 165 aa, Homo sapiens
MDIAPPRLICDSRVLERYLLEAKEAENITTGCAEHCSLNENITVPDSKVNLYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYTNFLRGKLKLYTGEACR

SEQ ID NO: 77 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaatattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcaggctagggaacaattacagttgcacgtggataaggcggtttctggcctgcgcagccttaccacgctgctccgtgcactgggtgcccaaaaagaagctatctcgccgcctgacgcggcctcagcaacgccgttacgcactattacagccgataccttccgtaaactgtttcgcgtctacgccaacttcttgcgtggcaaactgaaaatttatacgggtgaggcttgtcgctga

SEQ ID NO: 78 - protein, 165 aa, Homo sapiens
MDIAPPRLICDSRVLERYLLEAKEAENITTGCVEYCSLNENITVPDTKVNFYAWKRMEVGQQAVEVWQGLALLSEAVLRGQTLLVNSSQAREQLQLHVDKAVSGLRSLTTLLRALGAQKEAISPPDAASATPLRTITADTFRKLFRVYANFLRGKLKIYTGEACR

SEQ ID NO: 79 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtggagaacattgctcactgaacgagactattactgtaccggatacgaaagtcaacttttatgcctggaaacgaatggaagttggacaacaggcggtggaagtttggcaggggcttgccctgttgtcggaggcagtcctgcggggtcaaactttactggtaacttccagtcagtcttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaaatttatacgggtgaggcttgtcgctga

SEQ ID NO: 80 - protein, 165 aa, Homo sapiens
MDIAPPRLICDSRVLERYLLEAKEAENITTGCGEHCSLNETITVPDTKVNFYAWKRMEVGQQAVEVWQGLALLSEAVLRGQTLLVTSSQSWEPLQLHVDKAVSGLRSLTTLLRALGAQKEAISPPDAASAAPLRTITADTFRKLFRVYSNFLRGKLKIYTGEACR

SEQ ID NO: 81 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagtcttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 82 - protein, 165 aa, Homo sapiens
MDIAPPRLICDSRVLERYLLEAKEAETITTGCAEHCSLNENITVPDTKVNFYAWKRMEVGQQAVEVWQGLALLSEAVLRGQTLLVNSSQSWEPLQLHVDKAVSGLRSLTTLLRALGAQKEAISPPDAASAAPLRTITADTFRKLFRVYSNFLRGKLKLYTGEACR

SEQ ID NO: 83 - DNA, 498 bp, Homo sapiens
  CDS (1)...(498)
atggatatcgccccgccccgtctgatttgcgacagcagggtgctagaaagatacctgttcgaagcgaaagaggctgaaaatatcaccacaggctgtgaagaaaattgctcactgaacgagagtattactgtaccggatacgaaagtcaacttgtatgcctggaaacgaatggaagttggacaacaggcggtggaagtttggcaggggcttgccctgttgtcggaggcagtcctgcggggtcaaactttactggtaaattccagtcagccttgggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 84 - protein, 165 aa, Homo sapiens
MDIAPPRLICDSRVLERYLFEAKEAENITTGCEENCSLNESITVPDTKVNLYAWKRMEVGQQAVEVWQGLALLSEAVLRGQTLLVNSSQPWELLQLHVDKAVSGLRSLTTLLRALGAQKEAISPPDAASAAPLRTITADTFRKLFRVYSNFLRGKLKLYTGEACR

SEQ ID NO: 85 - DNA, 498 bp, Homo sapiens
  CDS (1)...(498)
atggatatcgccccgccccgtctgatttgcgacagcagggtgctagaaagatacctgctcgaagcgaaagaggctgaaattatcactacaggctgtgcagaacattgctcactgaacgagaatattactgtaccggatgcgaaagtcaacctatatgcctggaaacgaatggaagttggacaacaggcggtgaaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttatgcactattacagccgataccttccgtaaactgtttcgcgtctactccaacttcttgcgtggcaaactgaaactttatacgggtgaggcttgtcgctga

SEQ ID NO: 86 - protein, 165 aa, Homo sapiens
MDIAPPRLICDSRVLERYLLEAKEAEIITTGCAEHCSLNENITVPDAKVNLYAWKRMEVGQQAVKVWQGLALLSEAVLRGQTLLVISSQPWEPLQLHVDKAVSGLRSLTTLLRALGAQKEAISPPDAASAAPLCTITADTFRKLFRVYSNFLRGKLKLYTGEACR

SEQ ID NO: 87 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaatattgctcactgaacgagactattactgtaccggattcgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaactttatacgggtgaggcttgtcgctga

SEQ ID NO: 88 - protein, 165 aa, Homo sapiens
MDIAPPRLICDSRVLERYLLEAKEAENITTGCAEYCSLNETITVPDSKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYANFLRGKLKLYTGEACR

SEQ ID NO: 89 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgcagaacattgctcactgaacgagaatattactgtaccggattcgaaagtcaacatgtatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 90 - protein, 165 aa, Homo sapiens
MDIAPPRLICDSRVLERYLLEAKEAESITTGCAEHCSLNENITVPDSKVNMYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 91 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagttaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaattttatacgggtgaggcttgtcgctga

SEQ ID NO: 92 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYPNFLRGKLKFYTGEACR

SEQ ID NO: 93 - DNA, 498 bp, Homo sapiens
  CDS (1)...(498)
atggatatcgccccgccccgtctgatttgcgacagcagggtgctagaaagacacctgctcgaagcgaaagaggctgaaaatatcaccacaggctgtgcagaacattgctcactgaacgagaatattactgtaccggattcgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacaggtgaggcttgtcgctga

SEQ ID NO: 94 - protein, 165 aa, Homo sapiens
MDIAPPRLICDSRVLERHLLEAKEAENITTGCAEHCSLNENITVPDSKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 95 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaaaattgctcactgaacgagattattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagacttgggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 96 - protein, 165 aa, Homo sapiens
MDIAPPRLICDSRVLERYLLEAKEAENITTGCAENCSLNEIITVPDTKVNFYAWKRMEVGQQAVEVWQGLALLSEAVLRGQTLLVNSSQTWEQLQLHVDKAVSGLRSLTTLLRALGAQKEAISPPDAASAAPLRTITADTFRKLFRVYSNFLRGKLKLYTGEACR

SEQ ID NO: 97 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 98 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKFYTGEACR

SEQ ID NO: 99 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaacattgctcactgaacgagaatattactgtaccagatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 100 - protein, 165 aa, Homo sapiens
MDIAPPRLICDSRVLERYLLEAKEAEIITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKFYTGEACR

SEQ ID NO: 101 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaacattgctcactgaacgagattattactgtaccggatccgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 102 - protein, 165 aa, Homo sapiens
MDIAPPRLICDSRVLERYLLEAKEAENITTGCVEHCSLNEIITVPDPKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 103 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgtagaacattgctcactgaacgagactattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagccttgggaaccattacagttgcacgtggataaggcggtttctggcctgtgcagctttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 104 - protein, 165 aa, Homo sapiens
MDIAPPRLICDSRVLERYLLEAKEAESITTGCVEHCSLNETITVPDTKVNFYAWKRMEVGQQAVEVWQGLALLSEAVLRGQTLLVSSSQPWEPLQLHVDKAVSGLCSFTTLLRALGAQKEAISPPDAASAAPLRTITADTFRKLFRVYSNFLRGKLKLYTGEACR

SEQ ID NO: 105 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaaaattgctcactgaacgagattattactgtaccggattcgaaagtcaacttttatgcctggaaacgaatggaagttggacaacaggcggtggaagtttggcaggggcttgccctgttgtcggaggcagtcctgcggggtcaaactttactggtaaattccagtcagcctggggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 106 - protein, 165 aa, Homo sapiens
MDIAPPRLICDSRVLERYLLEAKEAENITTGCAENCSLNEIITVPDSKVNFYAWKRMEVGQQAVEVWQGLALLSEAVLRGQTLLVNSSQPGELLQLHVDKAVSGLRSLTTLLRALGAQKEAISPPDAASAAPLRTITADTFRKLFRVYSNFLRGKLKLYTGEACR

SEQ ID NO: 107 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtggagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 108 - protein, 165 aa, Homo sapiens
MDIAPPRLICDSRVLERYLLEAKEAEIITTGCGEDCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 109 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgaagaatattgctcactgaacgagaatattactgtaccggatacgaaagtcagcttgtatgcctggaaacgaatggaagttggacaacaggcggtggaagtttggcaggggcttgccctgttgtcggaggcagtcctgcggggtcaaactttactggtaaattccagtcagcctggggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaactttatacgggtgaggcttgtcgctga

SEQ ID NO: 110 - protein, 165 aa, Homo sapiens
MDIAPPRLICDSRVLERYLLEAKEAESITTGCEEYCSLNENITVPDTKVSLYAWKRMEVGQQAVEVWQGLALLSEAVLRGQTLLVNSSQPGELLQLHVDKAVSGLRSLTTLLRALGAQKEAISPPDAASAAPLRTITADTFRKLFRVYTNFLRGKLKLYTGEACR

SEQ ID NO: 111 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtggagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaagtttatacgggtgaggcttgtcgctga

SEQ ID NO: 112 - protein, 165 aa, Homo sapiens
MDIAPPRLICDSRVLERYLLEAKEAETITTGCGEDCSLNENITVPDTKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYANFLRGKLKVYTGEACR

SEQ ID NO: 113 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgcagaacattgctcactgaacgagaatattactgtaccggatacgaaagtcaacctttatgcctggaaacgaatggaagttggacaacaggcggtggaagtttggcaggggcttgccctgttgtcggaggcagtcctgcggggtcaaactttactggtaagttccagtcagccttgggaaccattacagttgcacgtggataaggcggtttctggcctgcgcagccttaccacgctgctccgtgcactgggtgcccaaaaagaagttatctcgccgcctgacgcggcctcagcagcgccgttacgcactattacagccgataccttccgtaaactgtttcgcgtctaccccaacttcttgcgtggcaaactgaaactttatacgggtgaggcttgtcgctga

SEQ ID NO: 114 - protein, 165 aa, Homo sapiens
MDIAPPRLICDSRVLERYLLEAKEAETITTGCAEHCSLNENITVPDTKVNLYAWKRMEVGQQAVEVWQGLALLSEAVLRGQTLLVSSSQPWEPLQLHVDKAVSGLRSLTTLLRALGAQKEVISPPDAASAAPLRTITADTFRKLFRVYPNFLRGKLKLYTGEACR

SEQ ID NO: 115 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgtagaatattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcaggctggggaacgattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 116 - protein, 165 aa, Homo sapiens
MDIAPPRLICDSRVLERYLLEAKEAEIITTGCVEYCSLNENITVPDTKVNFYAWKRMEVGQQAVEVWQGLALLSEAVLRGQTLLVNSSQAGERLQLHVDKAVSGLRSLTTLLRALGAQKEAISPPDAASAAPLRTITADTFRKLFRVYSNFLRGKLKLYTGEACR

SEQ ID NO: 117 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtggagaacattgctcactgaacgagactattactgtaccggattcgaaagtcaacgtttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaagtttatacgggtgaggcttgtcgctga

SEQ ID NO: 118 - protein, 165 aa, Homo sapiens
MDIAPPRLICDSRVLERYLLEAKEAESITTGCGEHCSLNETITVPDSKVNVYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKVYTGEACR

SEQ ID NO: 119 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagagtattactgtaccggattcgaaagtcaacttgtatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaactttatacgggtgaggcttgtcgctga

SEQ ID NO: 120 - protein, 165 aa, Homo sapiens
MDIAPPRLICDSRVLERYLLEAKEAENITTGCAEHCSLNESITVPDSKVNLYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYANFLRGKLKLYTGEACR

SEQ ID NO: 121 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaattttatacgggtgaggcttgtcgctga

SEQ ID NO: 122 - protein, 165 aa, Homo sapiens
MDIAPPRLICDSRVLERYLLEAKEAENITTGCAEDCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYTNFLRGKLKFYTGEACR

SEQ ID NO: 123 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagcctcgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 124 - protein, 165 aa, Homo sapiens
MDIAPPRLICDSRVLERYLLEAKEAEIITTGCAEHCSLNENITVPDTKVNFYAWKRMEVGQQAVEVWQGLALLSEAVLRGQTLLVNSSQPREPLQLHVDKAVSGLRSLTTLLRALGAQKEAISPPDAASAAPLRTITADTFRKLFRVYSNFLRGKLKLYTGEACR

SEQ ID NO: 125 - DNA, 498 bp, Homo sapiens
  CDS (1)...(498)
atggatatcgcctcgccccgtctgatttgcgacagcagggtgctagaaagatacctgctcgaagcgaaagaggctgaaagtatcaccacaggctgtgtagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 126 - protein, 165 aa, Homo sapiens
MDIASPRLICDSRVLERYLLEAKEAESITTGCVEDCSLNENITVPDTKVNFYAWKRMEVGQQAVEVWQGLALLSEAVLRGQTLLVSSSQPWEPLQLHVDKAVSGLRSLTTLLRALGAQKEAISPPDAASAAPLRTITADTFRKLFRVYSNFLRGKLKLYTGEACR

SEQ ID NO: 127 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaatattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaaatttatacgggtgaggcttgtcgctga

SEQ ID NO: 128 - protein, 165 aa, Homo sapiens
MDIAPPRLICDSRVLERYLLEAKEAEIITTGCAEYCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYPNFLRGKLKIYTGEACR

SEQ ID NO: 129 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaacattgctcactgaacgagaatattactgtaccggatacgaaagtcaacttttatgcctggaaacgaatggaagttggacaacaggcggtggaagtttggcaagggcttgccctgttgtcggaggcagtcctgcggggtcaaactttactggtaacttccagtcagccttgggaatcattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 130 - protein, 165 aa, Homo sapiens
MDIAPPRLICDSRVLERYLLEAKEAENITTGCVEHCSLNENITVPDTKVNFYAWKRMEVGQQAVEVWQGLALLSEAVLRGQTLLVTSSQPWESLQLHVDKAVSGLRSLTTLLRALGAQKEAISPPDAASAAPLRTITADTFRKLFRVYSNFLRGKLKLYTGEACR

SEQ ID NO: 131 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgaagaatattgctcactgaacgagaatattactgtaccggatgcgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 132 - protein, 165 aa, Homo sapiens
MDIAPPRLICDSRVLERYLLEAKEAENITTGCEEYCSLNENITVPDAKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 133 - DNA, 498 bp, Homo sapiens; identical to SEQ ID NO: 121
  CDS (1)...(498)

SEQ ID NO: 134 - protein, 165 aa, Homo sapiens; identical to SEQ ID NO: 122

SEQ ID NO: 135 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaatattgctcactgaacgagagtattactgtaccggattcgaaagtcaacttttatgcctggaaacgaatggaagttggacaacaggcggtggaagtttggcaggggcttgccctgttgtcggaggcagtcctgcggggtcaaactttactggtaagttccagtcagcctggggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaactttatacgggtgaggcttgtcgctga

SEQ ID NO: 136 - protein, 165 aa, Homo sapiens
MDIAPPRLICDSRVLERYLLEAKEAENITTGCAEYCSLNESITVPDSKVNFYAWKRMEVGQQAVEVWQGLALLSEAVLRGQTLLVSSSQPGEQLQLHVDKAVSGLRSLTTLLRALGAQKEAISPPDAASAAPLRTITADTFRKLFRVYANFLRGKLKLYTGEACR

SEQ ID NO: 137 - DNA, 498 bp, Homo sapiens
  CDS (1)...(498)
atggatatcgccccgccccgtctgatttgcgacagcaaggtgctagaaagatacctgctcgaagcgaaagaggctgaaattatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagacttgggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 138 - protein, 165 aa, Homo sapiens
MDIAPPRLICDSKVLERYLLEAKEAEIITTGCAEHCSLNENITVPDTKVNFYAWKRMEVGQQAVEVWQGLALLSEAVLRGQTLLVNSSQTWELLQLHVDKAVSGLRSLTTLLRALGAQKEAISPPDAASAAPLRTITADTFRKLFRVYSNFLRGKLKLYTGEACR

SEQ ID NO: 139 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaagattgctcactgaacgagactattactgtaccggatacgaaagtcaacttttatgcctggaaacgaatggaagttggacaacaggcggtggaagtttggcaggggcttgccctgttgtcggaggcagtcctgcggggtcaaactttactggtaacttccagtcaggctcgggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaaatttatacgggtgaggcttgtcgctga

SEQ ID NO: 140 - protein, 165 aa, Homo sapiens
MDIAPPRLICDSRVLERYLLEAKEAENITTGCVEDCSLNETITVPDTKVNFYAWKRMEVGQQAVEVWQGLALLSEAVLRGQTLLVTSSQAREQLQLHVDKAVSGLRSLTTLLRALGAQKEAISPPDAASAAPLRTITADTFRKLFRVYSNFLRGKLKIYTGEACR

SEQ ID NO: 141 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagagtattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 142 - protein, 165 aa, Homo sapiens
MDIAPPRLICDSRVLERYLLEAKEAENITTGCAEHCSLNESITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKFYTGEACR

SEQ ID NO: 143 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgcagaacattgctcactgaacgagaatattactgtaccggatacgaaagtcaacttttatgcccggaaacgaatggaagttggacaacaggcggtggaagtttggcaggggcttgccctgttgtcggaggcagtcctgcggggtcaaactttactggtaacttccagtcaggcttgggaacgattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaagtttatacgggtgaggcttgtcgctga

SEQ ID NO: 144 - protein, 165 aa, Homo sapiens
MDIAPPRLICDSRVLERYLLEAKEAETITTGCAEHCSLNENITVPDTKVNFYARKRMEVGQQAVEVWQGLALLSEAVLRGQTLLVTSSQAWERLQLHVDKAVSGLRSLTTLLRALGAQKEAISPPDAASAAPLRTITADTFRKLFRVYPNFLRGKLKVYTGEACR

SEQ ID NO: 145 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 146 - protein, 165 aa, Homo sapiens
MDIAPPRLICDSRVLERYLLEAKEAETITTGCAEHCSLNENITVPDTKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 147 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaacattgctcactgaacgagaatattactgtaccggatacgaaagtcaacttttatgcctggaaacgaatggaagttggacaacaggcggtggaagtttggcaggggcttgccctgttgtcggaggcagtcctgcagggtcaaactttactggtaaattccagtcagccttgggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 148 - protein, 165 aa, Homo sapiens
MDIAPPRLICDSRVLERYLLEAKEAENITTGCVEHCSLNENITVPDTKVNFYAWKRMEVGQQAVEVWQGLALLSEAVLQGQTLLVNSSQPWELLQLHVDKAVSGLRSLTTLLRALGAQKEAISPPDAASAAPLRTITADTFRKLFRVYSNFLRGKLKLYTGEACR

SEQ ID NO: 149 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagagtattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 150 - protein, 165 aa, Homo sapiens
MDIAPPRLICDSRVLERYLLEAKEAENITTGCAEHCSLNESITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 151 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaacattgctcactgaacgagaatattactgtaccggatgcgaaagtcaacgtatatgcctggaaacgaatggaagttggacaacaggcggtggaagtttggcaggggcttgccctgttgtcggaggcagtcctgcggggtcaaactttactggtaaattccagtcagtcttgggaaccattacagttgcacgtggataaggcggtttctggcctgcgcagccttaccacgctgctccgtgcactgggtgcccaaaaagaagctatctcgccgcctgacgcggcctcagcagcgccgttacacactattacagccgataccttccgtaaactgtttcgcgtctactccaacttcttgcgtggcaaactgaaactttatacgggtgaggcttgtcgctga

SEQ ID NO: 152 - protein, 165 aa, Homo sapiens
MDIAPPRLICDSRVLERYLLEAKEAEIITTGCAEHCSLNENITVPDAKVNVYAWKRMEVGQQAVEVWQGLALLSEAVLRGQTLLVNSSQSWEPLQLHVDKAVSGLRSLTTLLRALGAQKEAISPPDAASAAPLHTITADTFRKLFRVYSNFLRGKLKLYTGEACR

SEQ ID NO: 153 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagattattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcaggcttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaactttttgcgtggcaaactgaaactttatacgggtgaggcttgtcgctga

SEQ ID NO: 154 - protein, 165 aa, Homo sapiens
MDIAPPRLICDSRVLERYLLEAKEAENITTGCAEHCSLNEIITVPDTKVNFYAWKRMEVGQQAVEVWQGLALLSEAVLRGQTLLVNSSQAWEPLQLHVDKAVSGLRSLTTLLRALGAQKEAISPPDAASAAPLRTITADTFRKLFRVYSNFLRGKLKLYTGEACR

SEQ ID NO: 155 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggattcgaaagtcaacttttatgcctggaaacgaatggaagttggacaacaggcggtggaagtttggcaggggcttgccctgttgtcggaggcagtcctgcggggtcaaactttactggtaatttccagtcagcctggggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 156 - protein, 165 aa, Homo sapiens
MDIAPPRLICDSRVLERYLLEAKEAENITTGCAEHCSLNENITVPDSKVNFYAWKRMEVGQQAVEVWQGLALLSEAVLRGQTLLVISSQPGEPLQLHVDKAVSGLRSLTTLLRALGAQKEAISPPDAASAAPLRTITADTFRKLFRVYSNFLRGKLKLYTGEACR

SEQ ID NO: 157 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacgtatatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaactttatacgggtgaggcttgtcgctga

SEQ ID NO: 158 - protein, 165 aa, Homo sapiens
MDIAPPRLICDSRVLERYLLEAKEAENITTGCAEHCSLNENITVPDTKVNVYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYPNFLRGKLKLYTGEACR

SEQ ID NO: 159 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgaagaatattgctcactgaacgagactattactgtaccggatacgaaagtcaacttttatgcctggaaacgaatggaagttggacaacgggcggtggaagtttggcaggggcttgccctgttgtcggaggcagtcctgcggggtcaaactttactggtaatttccagtcagtctagggaacgattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 160 - protein, 165 aa, Homo sapiens
MDIAPPRLICDSRVLERYLLEAKEAENITTGCEEYCSLNETITVPDTKVNFYAWKRMEVGQRAVEVWQGLALLSEAVLRGQTLLVISSQSRERLQLHVDKAVSGLRSLTTLLRALGAQKEAISPPDAASAAPLRTITADTFRKLFRVYSNFLRGKLKLYTGEACR

SEQ ID NO: 161 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagactattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 162 - protein, 165 aa, Homo sapiens
MDIAPPRLICDSRVLERYLLEAKEAENITTGCAEHCSLNETITVPDTKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 163 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgaagaacattgctcactgaacgagattattactgtaccggatacgaaagtcaacttttatgcctggaaacgaatggaagttggacaacaggcggtggaagtttggcaggggcttgccctgttgtcggaggcagtcctgcggggtcaaactttactggtaacttccagtcagcctagggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaattttatacgggtgaggcttgtcgctga

SEQ ID NO: 164 - protein, 165 aa, Homo sapiens
MDIAPPRLICDSRVLERYLLEAKEAENITTGCEEHCSLNEIITVPDTKVNFYAWKRMEVGQQAVEVWQGLALLSEAVLRGQTLLVTSSQPREQLQLHVDKAVSGLRSLTTLLRALGAQKEAISPPDAASAAPLRTITADTFRKLFRVYTNFLRGKLKFYTGEACR

SEQ ID NO: 165 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaagattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 166 - protein, 165 aa, Homo sapiens
MDIAPPRLICDSRVLERYLLEAKEAENITTGCVEDCSLNENITVPDTKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKFYTGEACR

SEQ ID NO: 167 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgcaaaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 168 - protein, 165 aa, Homo sapiens
MDIAPPRLICDSRVLERYLLEAKEAETITTGCAKHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 169 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaatattgctcactgaacgagaatattactgtaccggatacgaaagtcaacttttatgcctggaaacgaatggaagttggacaacaggcggtggaagtttggcaggggcttgccctgttgtcggaggcagtcctgctgggtcaaactttactggtaacttccagtcagccttgggaatcattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaactttatacgggtgaggcttgtcgctga

SEQ ID NO: 170 - protein, 165 aa, Homo sapiens
MDIAPPRLICDSRVLERYLLEAKEAENITTGCAEYCSLNENITVPDTKVNFYAWKRMEVGQQAVEVWQGLALLSEAVLLGQTLLVTSSQPWESLQLHVDKAVSGLRSLTTLLRALGAQKEAISPPDAASAAPLRTITADTFRKLFRVYPNFLRGKLKLYTGEACR

SEQ ID NO: 171 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcccggaaacgaatggaagttggacaacaggcggtggaagtttggcaggggcttgccctgttgtcggaggcagtcctgcggggtccaactttactggtaacttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 172 - protein, 165 aa, Homo sapiens
MDIAPPRLICDSRVLERYLLEAKEAENITTGCAEHCSLNENITVPDTKVNFYARKRMEVGQQAVEVWQGLALLSEAVLRGPTLLVTSSQPWEPLQLHVDKAVSGLRSLTTLLRALGAQKEAISPPDAASAAPLRTITADTFRKLFRVYSNFLRGKLKLYTGEACR

SEQ ID NO: 173 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagactattactgtaccggattcgaaagtcaacctatatgcctggaaacgaatggaagttggacaacaggcggtggaagtttggcaggggcttgccctgttgtcggaggcagtcctgcggggtcaaaatttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaatctttatacgggtgaggcttgtcgctga

SEQ ID NO: 174 - protein, 165 aa, Homo sapiens
MDIAPPRLICDSRVLERYLLEAKEAENITTGCAEHCSLNETITVPDSKVNLYAWKRMEVGQQAVEVWQGLALLSEAVLRGQNLLVNSSQPWEPLQLHVDKAVSGLRSLTTLLRALGAQKEAISPPDAASAAPLRTITADTFRKLFRVYTNFLRGKLNLYTGEACR

SEQ ID NO: 175 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaacattgctcactgaacgagaatattactgtaccggattcgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaattttatacgggtgaggcttgtcgctga

SEQ ID NO: 176 - protein, 165 aa, Homo sapiens
MDIAPPRLICDSRVLERYLLEAKEAEIITTGCAEHCSLNENITVPDSKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYANFLRGKLKFYTGEACR

SEQ ID NO: 177 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatgcgaaagtcaacctatatgcctggaaacgaatggaagttggacaacaggcggtggaagtttggcaggggcttgccctgttgtcggaggcagtcctgcggggtcaaactttactggtaaattccagtcagtcttgggaacgattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 178 - protein, 165 aa, Homo sapiens
MDIAPPRLICDSRVLERYLLEAKEAENITTGCAEHCSLNENITVPDAKVNLYAWKRMEVGQQAVEVWQGLALLSEAVLRGQTLLVNSSQSWERLQLHVDKAVSGLRSLTTLLRALGAQKEAISPPDAASAAPLRTITADTFRKLFRVYSNFLRGKLKLYTGEACR

SEQ ID NO: 179 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgcagaacattgctcactgaacgagaatattactgtaccggattcgaaagtcaacatctatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggtaaactgaaactttatacgggtgaggcttgtcgctga

SEQ ID NO: 180 - protein, 165 aa, Homo sapiens
MDIAPPRLICDSRVLERYLLEAKEAESITTGCAEHCSLNENITVPDSKVNIYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 181 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgcagaacattgttcactgaacgagaatattactgtaccggatgcgaaagtcaacttgtatgcctggaaacgaatggaagttggacaacaggcggtggaagtttggcaggggcttgccctgttgtcggaggcagtcctgcggggtcaaactttactggtaaattccagtcagtcttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 182 - protein, 165 aa, Homo sapiens
MDIAPPRLICDSRVLERYLLEAKEAESITTGCAEHCSLNENITVPDAKVNLYAWKRMEVGQQAVEVWQGLALLSEAVLRGQTLLVNSSQSWEPLQLHVDKAVSGLRSLTTLLRALGAQKEAISPPDAASAAPLRTITADTFRKLFRVYSNFLRGKLKLYTGEACR

SEQ ID NO: 183 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtggagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaagtttatacgggtgaggcttgtcgctga

SEQ ID NO: 184 - protein, 165 aa, Homo sapiens
MDIAPPRLICDSRVLERYLLEAKEAESITTGCGEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYANFLRGKLKVYTGEACR

SEQ ID NO: 185 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgtagaaaattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagtctagggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaagtttatacgggtgaggcttgtcgctga

SEQ ID NO: 186 - protein, 165 aa, Homo sapiens
MDIAPPRLICDSRVLERYLLEAKEAENITTGCVENCSLNENITVPDTKVNFYAWKRMEVGQQAVEVWQGLALLSEAVLRGQTLLVNSSQSREPLQLHVDKAVSGLRSLTTLLRALGAQKEAISPPDAASAAPLRTITADTFRKLFRVYTNFLRGKLKVYTGEACR

SEQ ID NO: 187 - DNA, 498 bp, Homo sapiens
  CDS (1)...(498)
atggatatcgccccgccccgtctgatttgcgacagcagggtgctagaaagatacctgctcgaagcgaaagaggctgaaactatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagcctcgggaacgattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaagtttatacgggtgaggcttgtcgctga

SEQ ID NO: 188 - protein, 165 aa, Homo sapiens
MDIAPPRLICDSRVLERYLLEAKEAETITTGCAEHCSLNENITVPDTKVNFYAWKRMEVGQQAVEVWQGLALLSEAVLRGQTLLVSSSQPRERLQLHVDKAVSGLRSLTTLLRALGAQKEAISPPDAASAAPLRTITADTFRKLFRVYANFLRGKLKVYTGEACR

SEQ ID NO: 189 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgaagaagattgctcactgaacgagaatattactgtaccggatccgaaagtcaacatgtatgcctggaaacgaatggaagttggacaacaggcggtggaagtttggcaggggcttgccctgttgtcggaggcagtcctgcggggtcaaactttactggtaatttccagtcagtctagggaactattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacaccaacttcttgcgtggcaaactgaaactttatacgggtgaggcttgtcgctga

SEQ ID NO: 190 - protein, 165 aa, Homo sapiens
MDIAPPRLICDSRVLERYLLEAKEAESITTGCEEDCSLNENITVPDPKVNMYAWKRMEVGQQAVEVWQGLALLSEAVLRGQTLLVISSQSRELLQLHVDKAVSGLRSLTTLLRALGAQKEAISPPDAASAAPLRTITADTFRKLFRVYTNFLRGKLKLYTGEACR

SEQ ID NO: 191 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagactattactgtaccggatgcgaaagtcaacatttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 192 - protein, 165 aa, Homo sapiens
MDIAPPRLICDSRVLERYLLEAKEAENITTGCAEHCSLNETITVPDAKVNIYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 193 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcccggaaacgaatggaagttggacaacaggcggtggaagtttggcaggggcttgccctgttgtcggaggcagtcctgcggggtcaaactttactggtaacttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaaatttatacgggtgaggcttgtcgctga

SEQ ID NO: 194 - protein, 165 aa, Homo sapiens
MDIAPPRLICDSRVLERYLLEAKEAENITTGCAEHCSLNENITVPDTKVNFYARKRMEVGQQAVEVWQGLALLSEAVLRGQTLLVTSSQPWEPLQLHVDKAVSGLRSLTTLLRALGAQKEAISPPDAASAAPLRTITADTFRKLFRVYSNFLRGKLKIYTGEACR

SEQ ID NO: 195 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaagttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccattacgcactattacagccgataccttccgtaaactgtttcgcgtctactccaacttcttgcgtggcaaactgaaactttatacgggtgaggcttgtcgctga

SEQ ID NO: 196 - protein, 165 aa, Homo sapiens
MDIAPPRLICDSRVLERYLLEAKEAENITTGCAEHCSLNENITVPDTKVNFYAWKRMEVGQQAVEVWQGLALLSEAVLRGQTLLVSSSQPWEPLQLHVDKAVSGLRSLTTLLRALGAQKEAISPPDAASAAPLRTITADTFRKLFRVYSNFLRGKLKLYTGEACR

SEQ ID NO: 197 - DNA, 498 bp, Homo sapiens
  CDS (1)...(498)
atggatatcgccccgccccgtctgatttgcgacagcagggtgttagaaagatacctgttcgaagcgaaagaggctgaaaatatcaccacaggctgtgcagaatattgctcactgaacgagaatattactgtaccggatacgaaagtcaacttttatgcccggaaacgaatggaagttggacaacaggcggtggaagtttggcaggggcttgccctgttgtcggaggcagtcctgcggggtcaaactttactggtaatttccagtcagccttgggaaccattacagttgcacgtggataagtcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 198 - protein, 165 aa, Homo sapiens
MDIAPPRLICDSRVLERYLFEAKEAENITTGCAEYCSLNENITVPDTKVNFYARKRMEVGQQAVEVWQGLALLSEAVLRGQTLLVISSQPWEPLQLHVDKSVSGLRSLTTLLRALGAQKEAISPPDAASAAPLRTITADTFRKLFRVYSNFLRGKLKFYTGEACR

SEQ ID NO: 199 - DNA, 498 bp, Homo sapiens
  CDS (1)...(498)
atggatatcgccccgccccgtctgatttgcgacagcagggtgctagaaagatacctgctcgaagcgaaagaggctgaaattatcaccacaggctgtgcagaatattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 200 - protein, 165 aa, Homo sapiens
MDIAPPRLICDSRVLERYLLEAKEAEIITTGCAEYCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 201 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtggagaatattgctcactgaacgagaatattactgtaccggatacgaaagtcaacgtatatgcctgggaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaactttatacgggtgaggcttgtcgctga

SEQ ID NO: 202 - protein, 165 aa, Homo sapiens
MDIAPPRLICDSRVLERYLLEAKEAENITTGCGEYCSLNENITVPDTKVNVYAWERMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 203 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgcttactgaacgagattattactgtaccggattcgaaagtcaacttgtatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaattttatacgggtgaggcttgtcgctga

SEQ ID NO: 204 - protein, 165 aa, Homo sapiens
MDIAPPRLICDSRVLERYLLEAKEAENITTGCAEHCLLNEIITVPDSKVNLYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKFYTGEACR

SEQ ID NO: 205 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaacattgctcactgaacgagagtattactgtaccggatacgaaagtcaacctatatgcctggaaacgaatggaagttggacaacaggcggtggaagtttggcaggggcttgccctgttgtcggaggcagtcctgcggggtcaaactttactggtaagttccagtcagtctagggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaagtttatacgggtgaggcttgtcgctga

SEQ ID NO: 206 - protein, 165 aa, Homo sapiens
MDIAPPRLICDSRVLERYLLEAKEAENITTGCAEHCSLNESITVPDTKVNLYAWKRMEVGQQAVEVWQGLALLSEAVLRGQTLLVSSSQSREQLQLHVDKAVSGLRSLTTLLRALGAQKEAISPPDAASAAPLRTITADTFRKLFRVYSNFLRGKLKVYTGEACR

SEQ ID NO: 207 - DNA, 498 bp, Homo sapiens
  CDS (1)...(498)
atggatatcgccccgccccgtctgatttgcgacagcagggtgctagtaagatacctgctcgaagcgaaagaggctgaaattatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaaattccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctacgccaacttcttgcgtggcaaactgaaactttatacgggtgaggcttgtcgctga

SEQ ID NO: 208 - protein, 165 aa, Homo sapiens
MDIAPPRLICDSRVLVRYLLEAKEAEIITTGCAEHCSLNENITVPDTKVNFYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYANFLRGKLKLYTGEACR

SEQ ID NO: 209 - DNA, 498 bp, Homo sapiens
  CDS (1)...(498)
atggatatcgccccgccccgtctgatttgcgacagcagggtgctagaaagatacctgctcgaagcgaaagaggctgaaaatatcaccacaggctgtgcagaagattgctcactgaacgagagtattactgtaccggattcgaaagtcaacttatatgcctggaaacgaatggaagttggacaacaggcggtggaagtttggcaggggcttgccctgttgtcggaggcagtcctgcggggtcaaactttactggtaaattccagtcagcctggggaacaattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctactccaacttcttgcgtggcaaactgaaaatttatacgggtgaggcttgtcgctga

SEQ ID NO: 210 - protein, 165 aa, Homo sapiens
MDIAPPRLICDSRVLERYLLEAKEAENITTGCAEDCSLNESITVPDSKVNLYAWKRMEVGQQAVEVWQGLALLSEAVLRGQTLLVNSSQPGEQLQLHVDKAVSGLRSLTTLLRALGAQKEAISPPDAASAAPLRTITADTFRKLFRVYSNFLRGKLKIYTGEACR

SEQ ID NO: 211 - DNA, 498 bp, Homo sapiens
  CDS (1)...(498)
atggatatcgccccgccccgtctgatttgcgacagcagggtgctagaaagatacctgctcgaagcgaaagaggctgaaagtatcaccacaggctgtgcagaacattgctcactgaacgagaatattactgtaccggatacgaaagtcaacttttatgcctggaaacgaatggaagttggacaacaggcggtggaagtttggcaggggcttgccctgttgtcggaggcagtcctgcggggtcaaactttactggtaatttccagtcagccttgggaaccattacagttgcacgtggataaggcggtttctggcctgcgcagccttaccacgctgctccgtgcactgggtgcccaaaaagaagctatctcgccgcctgacgcggcctcagcagcgccgttacgcactattacagccgataccttccgtaaactgtttcgcgtctaccccaacttcttgcgtggcaaactgaaactttatacgggtgaggcttgtcgctga

SEQ ID NO: 212 - protein, 165 aa, Homo sapiens
MDIAPPRLICDSRVLERYLLEAKEAESITTGCAEHCSLNENITVPDTKVNFYAWKRMEVGQQAVEVWQGLALLSEAVLRGQTLLVISSQPWEPLQLHVDKAVSGLRSLTTLLRALGAQKEAISPPDAASAAPLRTITADTFRKLFRVYPNFLRGKLKLYTGEACR

SEQ ID NO: 213 - DNA, 1342 bp, Homo sapiens
  CDS (263)...(763)
cccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggagaacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaaaaaaaaaaaaaa

SEQ ID NO: 214 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALRAQKEAISPPDAASAAPLRTITADTFRKLFRVYSNFLRGKLKLYTGEACRTGDR

SEQ ID NO: 215 - DNA, 24 bp, Artificial Sequence
  Primer
caggaattctgtttggaaactgtc

SEQ ID NO: 216 - DNA, 24 bp, Artificial Sequence
  Primer
actctcataccatggaagcttgca

SEQ ID NO: 217 - DNA, 567 bp, Homo sapiens
  CDS (1)...(567)
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaccatcaccaccggctgcgtggaggactgcagcctgaacgagaacatcaccgtgcccgacaccaaggtgaacttctacgcccggaagcggatggaggtgggccagcaggccgtggagatctggcagggcctggccctgctgagcgaggccgtgctgcggggccagaccctgctggtgatcagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccgg

SEQ ID NO: 218 - protein, 189 aa, Homo sapiens
MGVHECPAWLWLLLSLLSLPLGLPVLGAPPRLICDSRVLERYLLEAKEAETITTGCVEDCSLNENITVPDTKVNFYARKRMEVGQQAVEIWQGLALLSEAVLRGQTLLVISSQPWEPLQLHVDKAVSGLRSLTTLLRALGAQKEAISPPDAASAAPLRTITADTFRKLFRVYSNFLRGKLKLYTGEACR

SEQ ID NO: 219 - DNA, 567 bp, Homo sapiens
  CDS (1)...(567)
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaccatcaccaccggctgcgtggaggactgcagcctgaacgagaacatcaccgtgcccgacaccaaggtgaacttctacgcccggaagcggatggaggtgggccagcaggccgtggagatctggcagggcctggccctgctgagcgaggccgtgctgcggggccagaccctgctggtgatcagcagccaggtgaacgagaccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccgg

SEQ ID NO: 220 - protein, 189 aa, Homo sapiens
MGVHECPAWLWLLLSLLSLPLGLPVLGAPPRLICDSRVLERYLLEAKEAETITTGCVEDCSLNENITVPDTKVNFYARKRMEVGQQAVEIWQGLALLSEAVLRGQTLLVISSQVNETLQLHVDKAVSGLRSLTTLLRALGAQKEAISPPDAASAAPLRTITADTFRKLFRVYSNFLRGKLKLYTGEACR

SEQ ID NO: 221 - DNA, 567 bp, Homo sapiens
  CDS (1)...(567)
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagagcatcaccaccggctgcgccgagcactgcagcctgaacgagaacatcaccgtgcccgacagcaaggtgaacatgtacgcctggaagcggatggaggtgggccagcaggccgtggaggtgtggcagggcctggccctgctgagcgaggccgtgctgcggggccagaccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccgg

SEQ ID NO: 222 - protein, 189 aa, Homo sapiens
MGVHECPAWLWLLLSLLSLPLGLPVLGAPPRLICDSRVLERYLLEAKEAESITTGCAEHCSLNENITVPDSKVNMYAWKRMEVGQQAVEVWQGLALLSEAVLRGQTLLVNSSQPWEPLQLHVDKAVSGLRSLTTLLRALGAQKEAISPPDAASAAPLRTITADTFRKLFRVYSNFLRGKLKLYTGEACR

SEQ ID NO: 223 - DNA, 567 bp, Homo sapiens
  CDS (1)...(567)
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagagcatcaccaccggctgcgccgagcactgcagcctgaacgagaacatcaccgtgcccgacagcaaggtgaacatgtacgcctggaagcggatggaggtgggccagcaggccgtggaggtgtggcagggcctggccctgctgagcgaggccgtgctgcggggccagaccctgctggtgaacagcagccaggtgaacgagaccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccgg

SEQ ID NO: 224 - protein, 189 aa, Homo sapiens
MGVHECPAWLWLLLSLLSLPLGLPVLGAPPRLICDSRVLERYLLEAKEAESITTGCAEHCSLNENITVPDSKVNMYAWKRMEVGQQAVEVWQGLALLSEAVLRGQTLLVNSSQVNETLQLHVDKAVSGLRSLTTLLRALGAQKEAISPPDAASAAPLRTITADTFRKLFRVYSNFLRGKLKLYTGEACR

SEQ ID NO: 225 - DNA, 81 bp, Homo sapiens
  CDS (1)...(81)
atgggcgtgcacgagtgccccgcctggctgtggctgctgctgagcctgctgagcctgcccctgggcctgcccgtgctgggc

SEQ ID NO: 226 - protein, 27 aa, Homo sapiens
MGVHECPAWLWLLLSLLSLPLGLPVLG

(19) United States
(12) Patent Application Publication
(10) Pub. No.: US 2004/0157293 A1
(43) Pub. Date: Aug. 12, 2004

(54) Enhanced variants of erythropoietin and methods of use

(76) Inventors: Glen A. Evans, San Marcos, CA (US); Sally Jewell, La Jolla, CA (US); Mark Ware, San Diego, CA (US)

Correspondence Address: Cathryn Campbell, McDERMOTT, WILL & EMERY, Suite 700, 4370 La Jolla Village Drive, San Diego, CA, 92122, US

(21) Appl. No.: 10/705,217

(22) Filed: Nov. 7, 2003

Related U.S. Application Data
(63) Continuation-in-part of application No. 10/291,847, filed on Nov. 8, 2002.

(51) Int. Cl. C07K 14/505

(52) U.S. Cl. 435/69.1; 530/399; 435/320.1; 435/325

(57) ABSTRACT

The invention provides a human erythropoietin polypeptide variant containing a human erythropoietin amino acid sequence having an amino acid difference in two or more different Epo-modification regions and an enhanced erythropoietin activity, or a functional fragment thereof. Also provided is a human erythropoietin polypeptide variant containing a human erythropoietin amino acid sequence having an amino acid difference in two or more different Epo-modification regions and a moderated erythropoietin activity, or a functional fragment thereof. The invention further provides a composition containing a human erythropoietin polypeptide variant having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, and a pharmaceutically acceptable medium.

This application is a continuation-in-part of U.S. application Ser. No. 10/291,847, filed Nov. 8, 2002, which is expressly incorporated in its entirety by reference.

### BACKGROUND OF THE INVENTION

This invention relates to molecular medicine and, more specifically to erythropoietin (EPO) variant polypeptides having beneficial therapeutic properties.

Erythropoietin is a glycoprotein hormone involved in the proliferation and differentiation of bone marrow erythroid progenitor cells. In this way erythropoietin controls the number of red blood cells in the circulation and hence the oxygen-carrying capacity of the blood. The peripheral red cell count is kept constant by a controlled feedback mechanism involving oxygen supply, erythropoietin secretion and erythropoiesis. However, the system can become unbalanced in conditions such as chronic renal disease, chronic inflammation and prematurity.

Naturally occurring erythropoietin is produced by the liver during fetal life and by the kidney of adults. Chronic renal disease can result in a decrease in the amount of erythropoietin which leads to anemia. Anemia can result in many debilitating symptoms such as tiredness, lethargy, muscle fatigue, and poor exercise capability. Recombinant erythropoietin, produced by genetic engineering methods involving the expression of erythropoietin from a transformed host cell, has used in the treatment of anemia resulting from chronic renal failure or from complications associated with chemotherapy. In addition, recombinant human erythropoietin is used as hormonal replacement therapy to correct various types of anemia and replenish the red cell count following hemorrhage or blood donation for autologous transfusion.

However, undesirable side effects of erythropoietin therapy have also been encountered. For example, increases in blood pressure and blood viscosities have been noted in some patients. Also, in some patients drastic increases in hematocrit, hemoglobin and the number of erythroid precursor cells have been observed while in other patients the increase in hematocrit is too low. For patients who respond poorly to erythropoietin, a non-physiological dose increase of erythropoietin is contraindicated because of possible immune reactions. In addition, increasing the amount of erythropoietin can increase non-specific effects of the polypeptide. One non-specific effect of erythropoietin is the simulation of megakaryoctes to form thrombocytes which can increase the risk of thrombosis.

An alternative to increasing the dosage of recombinant erythropoietin is to generate new versions of the erythropoietin polypeptide that have altered activities compared to human erythropoietin. Such altered activities can include, for example, greater or lesser potency, or a faster or slower time of onset of an erythropoietin activity.

Thus, there exists a need for a human erythropoietin variant polypeptide with an altered erythropoietin activity which can be used, for example, as a therapeutic agent. The present invention satisfies this need and provides related advantages as well.

### SUMMARY OF THE INVENTION

The invention provides a human erythropoietin polypeptide variant containing a human erythropoietin amino acid sequence having an amino acid difference in two or more different Epo-modification regions and an enhanced erythropoietin activity, or a functional fragment thereof. Also provided is a human erythropoietin polypeptide variant containing a human erythropoietin amino acid sequence having an amino acid difference in two or more different Epo-modification regions and a moderated erythropoietin activity, or a functional fragment thereof. The invention further provides a composition containing a human erythropoietin polypeptide variant having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74,76,82,88,90,92,94, 96,98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, and a pharmaceutically acceptable medium.

### BRIEF DESCRIPTION OF THE DRAWINGS

FIG. 1 shows a chart containing erythropoietin polypeptide variants with SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 218, 220, 222, 224 referred to herein for convenience as SEQ ID NOS: 2-212 and 218-224 EVEN. The top line shows the amino acid sequence of human erythropoietin polypeptide variant SEQ ID NO:2. For the remaining erythropoietin polypeptide variants, an open box indicates the same amino acid as in SEQ ID NO:2 while a filled box indicates the single letter code of an amino acid difference from SEQ ID NO:2.

FIG. 2 shows a chart containing erythropoietin polypeptide variants with SEQ ID NOS:214, 218, 220, 222 and 224, following the drawing indications shown set forth above for FIG. 1.

### DETAILED DESCRIPTION OF THE INVENTION

This invention is directed to a human erythropoietin polypeptide variant or variants having an altered erythropoietin activity. These human erythropoietin polypeptide variants are advantageous in that can have desirable features when compared to the human erythropoietin polypeptide. For example, the human erythropoietin polypeptide variants of the invention can have increased or decreased erythroid precursor proliferation activity compared to the human erythropoietin polypeptide shown as SEQ ID NO:214. In addition, for example, the human erythropoietin polypeptide variants of the invention can be faster-acting or slower-acting when compared to the human erythropoietin polypeptide. Erythropoietin polypeptide variants of differing levels of activity or times to reach a maximal level of activity can be useful in the clinic in order to customize the level of erythropoietin activity to the needs of a particular individual. For example, an individual who does not respond to recombinant human erythropoietin therapy, or who responds sub-optimally, can respond to therapy with a more active human erythropoietin polypeptide variant. The invention therefore provides human erythropoietin polypeptide variants having altered erythropoietin activities which can be used therapeutically.

Even when therapy with recombinant human erythropoietin (rHuEPO) or a variant of rHuEPO called novel erythropoiesis-stimulating protein (NESP) (also known as darbepoetin or Aranesp™), has been effective for some individuals in the clinic, adverse side-effects of these therapies have been reported. For example, stimulating megakaryocytes to form thrombocytes is a side effect of erythropoietin therapy which occurs in some individuals. In this case a risk of thrombosis can arise during the treatment with erythropoietin, and the treatment must be stopped immediately. Here a higher specificity erythropoietin polypeptide variant would be desirable.

In addition, in a published study adverse events occurring in greater than 10% of individuals in either the NESP or rHuEO groups were hypertension, peripheral oedema, fatigue, diarrhea, headache, nausea and pruritus (Locatelli et al., Kidney International 60:741-747 (2001)). Some of these events can be attributed to concurrent medical conditions, however adverse events considered to be treatment-related were reported. Hypertension was the most common of these events. Another side-effect that has been reported in connection with certain forms of recombinant erythropoietin therapy is red-cell aplasia. Red-cell aplasia can result in the generation of antibodies in a subject that neutralize not only the drug, but also the individual's own natural erythropoietin. Therefore, there is a need for human erythropoietin polypeptide variants having altered erythropoietin activities which would reduce the number of side-effects of erythropoietin therapy.

In one embodiment, the invention is directed to a human erythropoietin polypeptide variant containing a human erythropoietin amino acid sequence having an amino acid difference in two or more different Epo-modification regions and an enhanced erythropoietin activity, or a functional fragment thereof. In another embodiment, the invention is directed to a human erythropoietin polypeptide variant containing a human erythropoietin amino acid sequence having an amino acid difference in two or more different Epo-modification regions and a moderated erythropoietin activity, or a functional fragment thereof. In a further embodiment, the invention is directed to a human erythropoietin polypeptide variant having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, or a functional fragment thereof.

As used herein, a “variant” when used in reference to a human erythropoietin polypeptide is intended to mean an erythropoietin polypeptide having a non-naturally occurring amino acid sequence that differs at one or more amino acid positions from the sequence of human erythropoietin. Human erythropoietin, as used herein, is the polypeptide shown in SEQ ID NO:214. The amino acid sequence shown in SEQ ID NO:214 is derived from the sequence for human erythropoietin referenced in GenBank by accession number NP_{—}000790. The only difference is the absence of a 27 amino acid leader sequence in SEQ ID NO:214 compared to accession number NP_{—}000790.

Variants differ from a human erythropoietin polypeptide by some detectable structural property such as a difference in at least one amino acid residue or a difference introduced by the modification of an amino acid such as the addition of a chemical functional group. A difference in an amino acid at a particular position also includes, for example, the insertion or deletion of an amino acid at that position. A human polypeptide variant can contain, for example, 1, 2, 3, 4, 5, 6, 7, 8, 9, 10, or 11 amino acid differences compared to human erythropoietin. In addition, for example, a human polypeptide variant can contain at least 1%, at least 2%, at least 3%, at least 4%, at least 5% or at least 6% amino acid differences compared to human erythropoietin.

As understood by one skilled in the art, a human erythropoietin polypeptide variant can contain additional sequences such as non-homologous sequences. For example, a human erythropoietin polypeptide variant can contain an epitope tag at the amino or carboxy termini of the polypeptide. Also, for example, a human erythropoietin polypeptide variant can be fused to other polypeptides such as gluthionine S-transferase (GST).

As used herein the term “Epo-modification region” is intended to mean a region of the human erythropoietin polypeptide SEQ ID NO:214 that is associated with or has an activity directed to the carbohydrate content of the polypeptide, aggregation properties of the polypeptide, or erythropoietin receptor (EPOR) binding by the polypeptide as described herein. For example, an Epo-modification region can contain amino acids associated with or having an activity directed to the carbohydrate content of erythropoietin described herein as, A30, H32, P87, W88, P90; amino acids associated with or having an activity directed to aggregation of erythropoietin described herein as N24, N38, N83; and amino acids associated with or having an activity directed to binding to EPOR described herein as T44, F48, N147, and L155.

The term “Epo-modification region” can be sub-divided into three different groups; the carbohydrate content region, the aggregation region, and the EPOR binding region. Therefore, the term “two or more different Epo-modification regions” refers to two or more of these three different Epo-modification regions. As described above, the carbohydrate content region includes amino acids associated with or having an activity directed to the carbohydrate content of erythropoietin such as, A30, H32, P87, W88, P90. The aggregation region includes amino acids associated with or having an activity directed to aggregation of erythropoietin such as N24, N38, N83. The EPOR binding region includes amino acids associated with or having an activity directed to binding to EPOR such as T44, F48, N147, and L155.

As used herein the term “amino acid difference” in reference to an Epo-modification region is intended to mean a difference between the amino acid sequence of a human erythropoietin polypeptide variant in the identified Epo-modification regions when compared to the amino acid sequence of human erythropoietin SEQ ID NO:214. A difference in amino acid sequence includes, for example, the substitution of one amino acid for another amino acid or the insertion or deletion of an amino acid. For example, a human erythropoietin polypeptide variant referenced herein as SEQ ID NO:90 has an amino acid difference in an Epo-modification region associated with aggregation (amino acid N24 is changed to S24) and an amino acid difference in an Epo-modification region associated with EPOR binding (amino acid T44 is changed to S44). SEQ ID NO:90 also contains a third mutation in an Epo-modification region associated with EPOR binding where amino acid F48 is changed to M48. These amino acid differences in Epo-modification regions also are present in versions of SEQ ID NO:90 that include a leader sequence directing secretion in mammalian cells. These versions are described further below and referenced herein as SEQ ID NOS:222 and 224. A difference in amino acid sequence can also include, for example, the inclusion of a modified amino acid instead of the naturally occurring amino acid at a particular location.

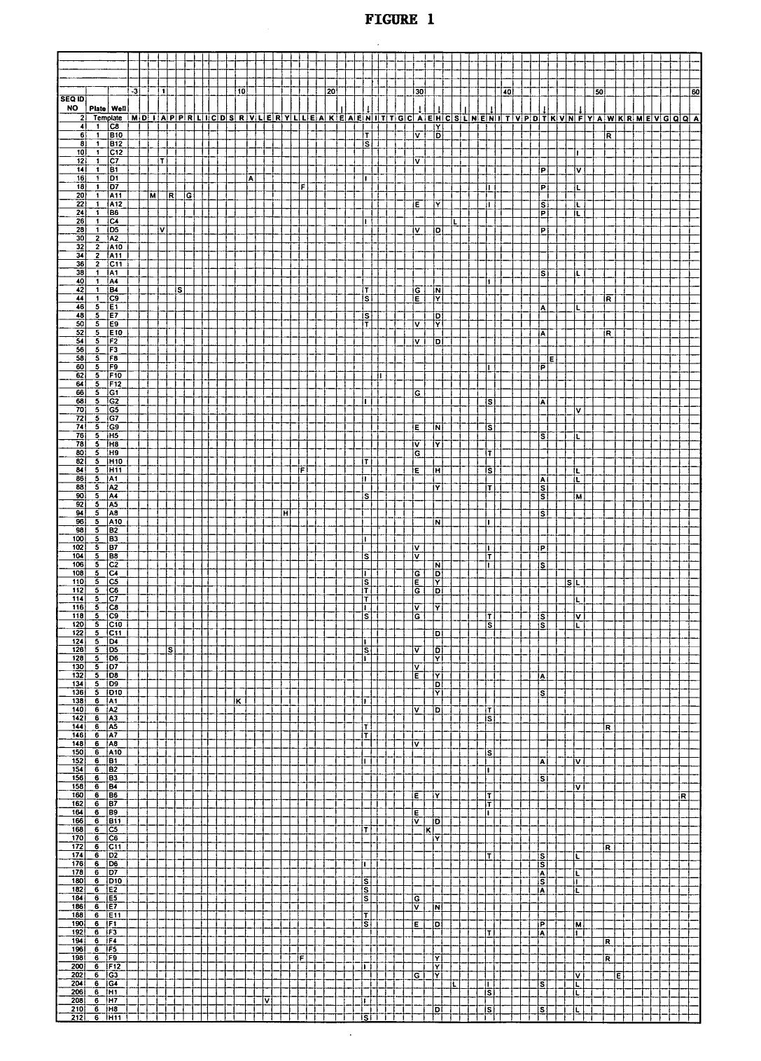

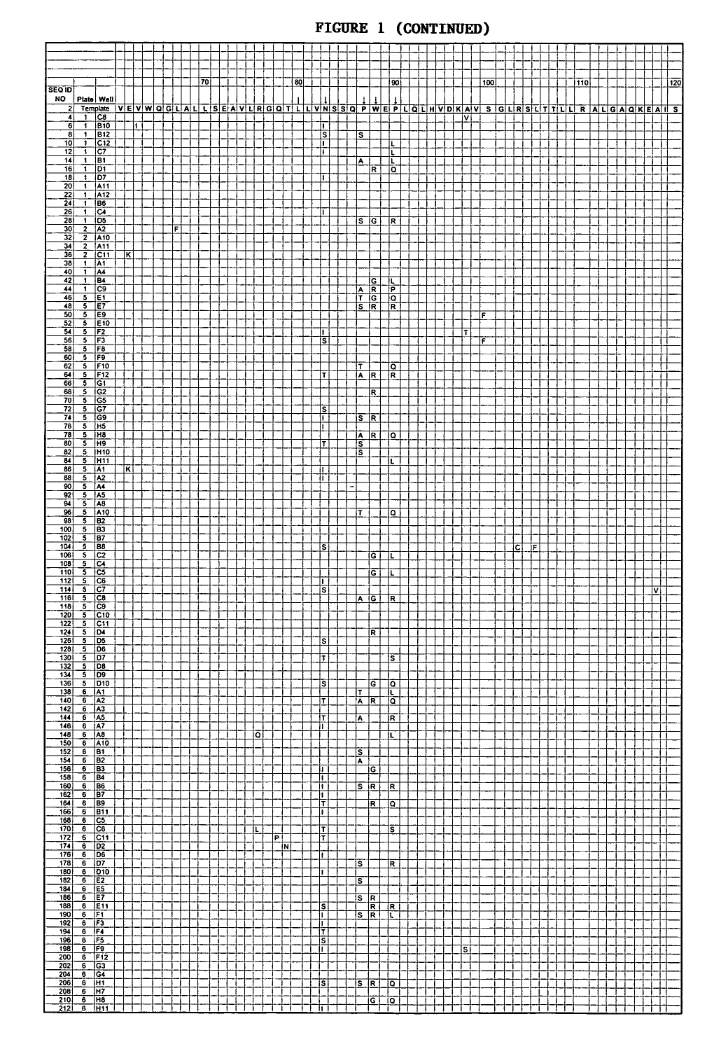

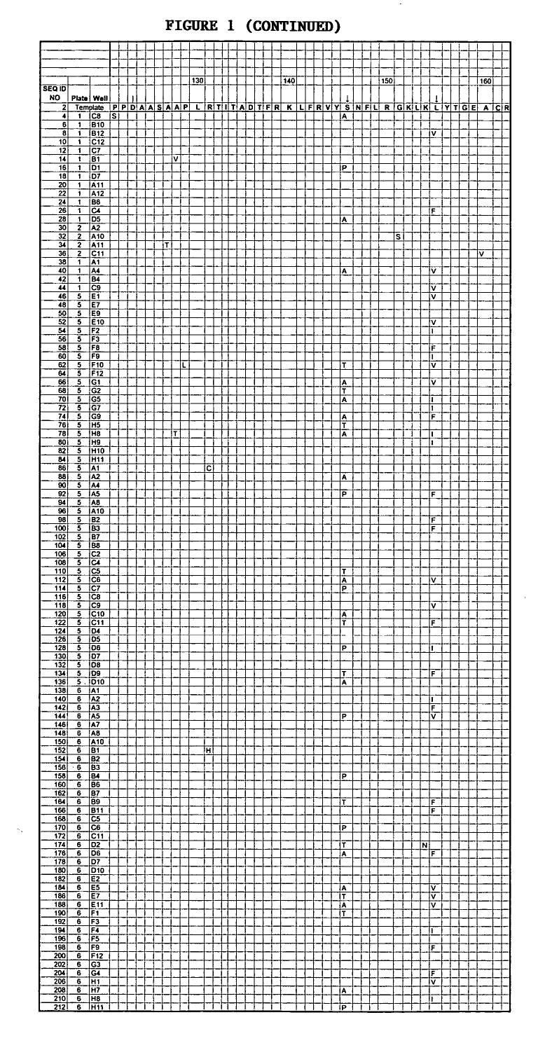

As shown in FIG. 1, the sequence used as a template for the generation of erythropoietin polypeptide variants herein, disclosed as SEQ ID NO:2, contains no amino acid differences in Epo-modification regions compared to the amino acid sequence of human erythropoietin SEQ ID NO:214. The polypeptide referenced as SEQ ID NO:2 does have amino acid differences compared to SEQ ID NO:214 outside of the Epo-modification regions, for example the substitution of an alanine at amino acid position 79 in SEQ ID NO:2 compared to threonine in SEQ ID NO:214.

As used herein, the term “functional fragment” is intended to mean a portion of a full-length human erythropoietin polypeptide variant or nucleic acid molecule where the portion retains an erythropoietin activity. For example, a functional fragment of a human erythropoietin polypeptide variant can be a fragment with the ability to increase erythroid precursor proliferation. Fragments can include, for example, amino terminal, carboxyl terminal, or internal deletions of a human erythropoietin polypeptide variant. For example, a fragment can contain at least about 25, 50, 75, 100, 125, or 150 contiguous or non-contiguous amino acid residues of a human erythropoietin polypeptide variant where the fragment retains an erythropoietin activity.

As used herein, the term “functional fragment” when used in reference to an antibody is intended to refer to a portion of an antibody which still retains some or all of the erythropoietin or erythropoietin variant binding activity. Such functional fragments can include, for example, antibody functional fragments such as Fv, single chain Fv (scFv), Fab, F(ab′), F(ab)2, F(ab′)2, and minibody. Other functional fragments can include, for example, heavy or light chain polypeptides, variable region polypeptides, CDR polypeptides, single domain antibodies, or portions thereof so long as such functional fragments retain binding activity.

As used herein, an “erythropoietin activity” is intended to mean an activity associated with a human erythropoietin polypeptide. In addition, the term an “erythropoietin activity” includes temporal qualities of the activity such as the time it takes to reach a maximal level of an erythropoietin activity or the time of onset of an erythropoietin activity. An erythropoietin activity can be a directly associated with a human erythropoietin polypeptide or indirectly associated with a human erythropoietin polypeptide. An activity directly associated with a human erythropoietin polypeptide can include, for example, the induction of erythroid precursor proliferation or the induction of erythroid precursor cell differentiation. An activity indirectly associated with a human erythropoietin polypeptide can include a result of an activity directly associated with an erythropoietin polypeptide such as, for example, an increased number of erythrocytes in an individual, an increased hematocrit in an individual, or reduction in anemia in an individual.

An erythropoietin activity can be an activity that is measured, for example, in vitro or in vivo. For example, an erythropoietin activity such as the induction of erythroid precursor cell differentiation can be measured in vitro using a bioassay in a cell line or in vivo by measuring hematocrit in a test animal. Different properties of an erythropoietin polypeptide can affect activity in vivo compared to in vitro. For example, the plasma half-life of a polypeptide can affect activity in vivo, but this property can have less effect on activity in vitro.

As used herein the term “enhanced erythropoietin activity” is intended to mean an increase in an erythropoietin activity or a faster time of an erythropoietin activity compared to the same activity or time of activity from human erythropoietin SEQ ID NO:214. For example, an enhanced erythropoietin activity can be an increase in erythroid precursor proliferation or a faster time to reach a maximal level of erythroid precursor proliferation. An enhanced erythropoietin activity can be measured, for example, in vitro or in vivo.

As understood by one skilled in the art, different preparations or sources of human erythropoietin SEQ ID NO:214 are available. For example, a source of human erythropoietin can be a commercially available form such as is available from Amgen. In addition, for example, a source of human erythropoietin can be the World Health Organization Second International Reference Preparation.

Furthermore, a polypeptide variant with essentially identical activity as human erythropoietin referenced as SEQ ID NO:214 can be used as a surrogate for human erythropoietin in subsequent assays. For example, as disclosed herein, the erythroid precursor proliferation activity of the polypeptide variant referenced as SEQ ID NO:2 is essentially identical to that of human erythropoietin obtained commercially from Amgen. Therefore, comparison of human erythropoietin polypeptide variants to the SEQ ID NO:2 polypeptide variant can be used to determine whether a human erythropoietin polypeptide variant has altered erythrocyte precursor proliferation activity compared to human erythropoietin.

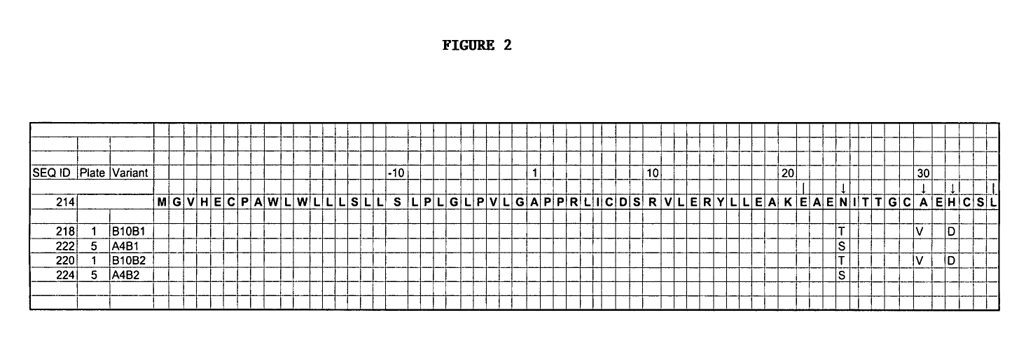

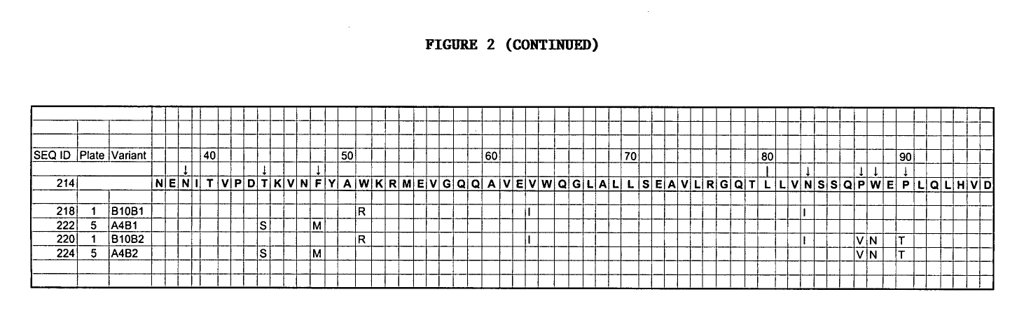

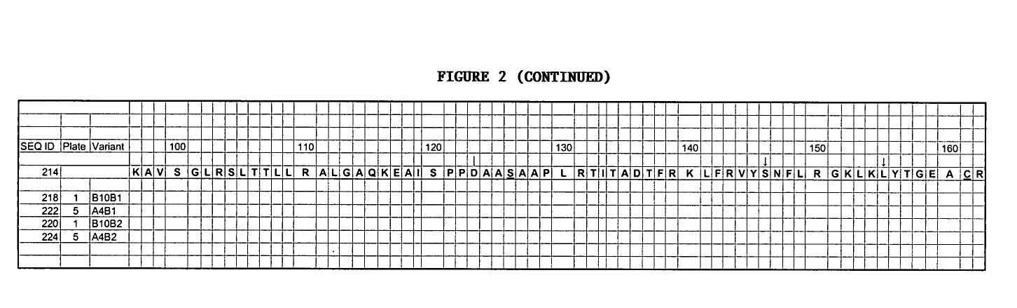

As disclosed herein, specific examples of a human erythropoietin polypeptide variant with an enhanced activity are the polypeptides referenced as SEQ ID NOS:90, 222, 224 (FIGS. 1 and 2). For example, the polypeptides referenced as SEQ ID NOS:90, 222, 224 show increased activity in an erythrocyte precursor proliferation assay compared to the polypeptide referenced as SEQ ID NO:2 (Table 1). Another example of a human erythropoietin polypeptide variant disclosed herein with an enhanced activity is the polypeptide referenced as SEQ ID NO:96 (FIG. 1). For example, the polypeptide referenced as SEQ ID NO:96 shows a faster time to reach a maximal level of activity in an erythrocyte precursor proliferation assay compared to the polypeptide referenced as SEQ ID NO:2 (Table 1).

As used herein the term “moderated erythropoietin activity” is intended to mean a decrease in an erythropoietin activity or a slower time of an erythropoietin activity compared to the same activity or time of activity from human erythropoietin SEQ ID NO:214. For example, a moderated erythropoietin activity can be a decrease in erythroid precursor proliferation or a slower time to reach a maximal level of erythroid precursor proliferation. A moderated erythropoietin activity can be measured, for example, in vitro or in vivo.

As disclosed herein, a specific example of a human erythropoietin polypeptide variant with a moderated activity is the polypeptide referenced as SEQ ID NO:198 (FIG. 1). For example, the polypeptide referenced as SEQ ID NO:198 shows decreased activity in an erythrocyte precursor proliferation assay compared to the polypeptide referenced as SEQ ID NO:2 (Table 1). Another example of a human erythropoietin polypeptide variant disclosed herein with a moderated activity is the polypeptide referenced as SEQ ID NO:8 (FIG. 1). For example, the polypeptide referenced as SEQ ID NO:8 shows a slower time to reach a maximal level of activity in an erythrocyte precursor proliferation assay compared to the polypeptide referenced as SEQ ID NO:2 (Table 1).

In addition, a human erythropoietin polypeptide variant can have an enhanced activity in regard to one type of activity an a moderated activity in regard to another type of activity. For example, the polypeptide referenced as SEQ ID NO:212 shows increased activity in an erythrocyte precursor proliferation assay compared to the polypeptide referenced as SEQ ID NO:2, but a slower time to reach a maximal level of activity in an erythrocyte precursor proliferation assay compared to the polypeptide referenced as SEQ ID NO:2 (Table 1). Another example of a human erythropoietin polypeptide variant disclosed herein with both an enhanced and moderated activity is the polypeptide referenced as SEQ ID NO:70 (FIG. 1). For example, the polypeptide referenced as SEQ ID NO:70 shows a decreased activity in an erythrocyte precursor proliferation assay compared to the polypeptide referenced as SEQ ID NO:2, but a faster time to reach a maximal level of activity in an erythrocyte precursor proliferation assay compared to the polypeptide referenced as SEQ ID NO:2 (Table 1).

As used herein, “effective amount” is intended to mean an amount sufficient to produce a desired effect. For example, when used in reference to a composition containing a human erythropoietin polypeptide variant it is intended to mean an amount of the composition sufficient to induce a therapeutic effect. For example, an effective amount of a composition, such as a pharmaceutical composition, containing a human erythropoietin polypeptide variant can be a range of protein concentrations correlated to the weight of an individual. For example, an effective amount can be at least 0.01 mg/kg, at least 0.05 mg/kg, at least 0.10 mg/kg, at least 0.25 mg/kg, at least 0.50 mg/kg, at least 0.75 mg/kg, at least 1.0 mg/kg, at least 1.5 mg/kg, at least 2.0 mg/kg, or more of a composition.

As used herein, the term “neurological condition” is intended to mean a pathological condition affecting neuronal or glial cells in the nervous system. Pathological conditions affecting neuronal or glial cells include ischemia, apoptosis, necrosis, oxidative or free radical damage, and excitotoxicity. For example, neurological conditions include cerebral and spinal ischemia, acute brain injury, spinal cord injury, retinal disease, and neurodegenerative diseases such as Alzheimer's disease, Parkinson's disease, Huntington's disease, and ALS.

Human erythropoietin is an acidic glycoprotein hormone with a molecular mass of 34 kD. As a regulator of red cell production, it functions to promote erythroid proliferation and differentiation and to initiate hemoglobin synthesis. Information regarding erythropoietin can be found, for example, at the National Center for Biotechnology Information website at OMIM entry number 133170 (URL: www.ncbi.nlm.nih.gov/htbin-post/Omim/dispmim?133170).

The human erythropoietin cDNA was first cloned in E. coli by Lee-Huang (Lee-Huang, S. Proc. Nat. Acad. Sci. 81:2708-2712 (1984); see also U.S. Pat. No. 4,703,008). McDonald et al., and Shoemaker and Mitsock cloned the mouse gene and the latter investigators showed that the nucleotide and amino acid sequence of erythropoietin are about 80% conserved between human and mouse (McDonald, et al. Mol. Cell. Biol. 6:842-848 (1986); Shoemaker and Mitsock, Mol. Cell. Biol. 6: 849-858 (1986)). This is a higher order of conservation than found in related polypeptides such as various interferons, interleukin-2, and GM-CSF. In addition, Sherwood and Shouval described a human renal carcinoma cell line that continuously produces erythropoietin (Sherwood and Shouval, Proc. Nat. Acad. Sci. 83:165-169 (1986)).

The erythropoietin gene has 5 exons that code for a 193-amino acid pro-polypeptide. A 27-amino acid leader sequence is cleaved off the amino terminus of the pro-polypeptide, yielding the functional 166-amino acid polypeptide. However, recombinant human erythropoietin expressed in Chinese hamster ovary cells contains only 165 amino acids, having lost arg166. The mechanism for this is undefined, and whether erythropoietin circulating in the plasma also lacks arg166 is not known. Both the nucleotide and amino acid sequences of erythropoietin are highly conserved among mammals.

Law et al. assigned erythropoietin to chromosome 7 by Southern blot analysis of DNA from human/Chinese hamster cell hybrids with a cDNA clone for the entire coding region of the gene (Law et al., Proc. Nat. Acad. Sci. 83:6920-6924 (1986)). Further localization to 7q11-q22 was achieved by in situ hybridization. They found a restriction fragment length polymorphism (RFLP) with a frequency of about 20% in a Chinese population. Using somatic cell hybrid analysis, Watkins et al., placed erythropoietin on the proximal half of 7q, closely linked to COL1A2 (see OMIM Entry 120160) and to DNA markers linked to CF (see OMIM entry 219700). Because of the close linkage of erythropoietin to COL1A2 and markers linked to CF, it is can be justified to narrow the assignment of erythropoietin to 7q21 -q22 (Watkins, et al., Cytogenet. Cell Genet. 42:214-218 (1986)). By in situ hybridization and genetic analysis using RFLPs in interspecific mouse backcross DNAs, Lacombe et al. demonstrated that erythropoietin is located on chromosome 5 in the mouse (Lacombe et al., (Letter) Blood 72:1440-1442 (1988)).

Synthesis of erythropoietin in the kidney and liver in response to hypoxia depends on both protein synthesis and heme synthesis. Goldberg et al. proposed a model in which a ligand-dependent conformational change in a heme protein accounts for the mechanism by which hypoxia as well as cobalt and nickel stimulates the production of erythropoietin (Goldberg et al., Science 242:1412-1415 (1988)). Semenza et al. generated transgenic mice containing the human erythropoietin gene and found increased erythropoietin mRNA expression not only in liver and kidney, but in all other transgenic tissues analyzed (Semenza et al., (Abstract) Am. J. Hum. Genet. 45 (suppl.):A1 16 only (1989); Semenza et al., Proc. Nat. Acad. Sci. 86:2301-2305 (1989)). The mice were polycythemic, with increased erythroid precursors in hematopoietic tissues and increased erythrocytic indices in peripheral blood. From further studies in these transgenic mice, Semenza et al. concluded that different DNA sequences flanking the erythropoietin gene control liver versus kidney expression of the gene and that some of these sequences are located 3-prime to the gene.

Erythropoiesis occurs in 2 distinct waves during embryogenesis: the primitive wave in the extraembryonic yolk sac followed by the definitive wave in the fetal liver and spleen. Even though progenitors for both cells types are present in the yolk sac blood islands, only primitive cells are formed in the yolk sac during early embryogenesis. Lee et al. presented results that led them to propose that erythropoietin expression and the resultant erythropoietin receptor activation regulate the timing of the definitive wave (Lee et al., Blood 98:1408-1415 (2001)). They demonstrated that erythropoietin and EPOR gene expression are temporally and spatially segregated: though EPOR is expressed early (embryonic days 8.0-9.5) in the yolk sac blood islands, no erythropoietin expression could be detected in this extraembryonic tissue. Only at a later stage can Erythropoietin expression be detected intraembryonically, and the onset of erythropoietin expression correlates with the initiation of definitive erythropoiesis. By ‘knocking in’ a constitutively active form of EPOR, R129C, they demonstrated further that the activation of the EPOR signaling pathway can lead to earlier onset of definitive erythropoiesis in the yolk sac. The observations provided insight into the in vivo mechanism by which two erythroid progenitor populations can coexist in the yolk sac yet always differentiate successively during embryogenesis.

Eschbach et al. demonstrated the effectiveness of recombinant human erythropoietin in treating the anemia of end-stage renal disease (Eschbach et al., New Eng. J. Med. 316:73-78 (1987)). Recombinant human erythropoietin has also been approved for the treatment of anemia associated with cancer, HIV infection, and use in the surgical setting to decrease the need for allogeneic blood transfusions. In addition to its role as a kidney cytokine regulating hematopoiesis, erythropoietin is also produced in the brain after oxidative or nitrosative stress. The role of erythropoietin in neurological conditions will be discussed further below.

Site-directed mutagenesis studies of human erythropoietin have identified several regions that are required for activity. For example, Elliot et al., have identified four regions to be important for bioactivity: amino acids 11 to 15, 44 to 51, 100 to 108, and 147 to 151 (Elliott et al., Blood 89:493-502 (1997)). Several mutants have been generated that have reduced or no activity when compared to human erythropoietin, however fewer mutants have been generated that have increased activity.

One mutant form of erythropoietin with enhanced activity in vivo is novel erythropoiesis-stimulating protein (NESP). The amino acid sequence of NESP differs from that of human erythropoietin at 5 positions (Ala30Asn, His32Thr, Pro87Val, Trp88Asn, and Pro90Thr) allowing for additional oligosaccharide attachment at asparagine residues at positions 30 and 88 (Elliott et al, Blood 96:82a (2000)). NESP is distinct from erythropoietin in that it has additional sialic acid which has been shown to confer an increased terminal half-life in animal models, patients with chronic renal failure, and cancer patients receiving multiple cycles of chemotherapy (Macdougall, et al., J. Am. Soc. Nephrol. 10:2392-2395 (1999)). In studies of 89 patients with non-myeloid malignancies, Smith et al. found that NESP was tolerated, with response rates ranging from 61 to 83%, depending on dosage (Smith et al., Brit. J. Cancer 84:24-30 (2001)).

One way to increase the activity of erythropoietin in vivo, is to increase the plasma half-life of the polypeptide in vivo. This can be accomplished, for example, by increasing the size of the polypeptide. For example, erythropoietin monomers can be chemically modified to form dimers and trimers. Indeed, dimerized and trimerized forms of human erythropoietin have been found to have increased plasma half-life and increased in vivo activity (Sytkowski et al., Proc. Natl. Acad. Sci. USA 95:1184-1188 (1998)).

As disclosed herein, several human erythropoietin polypeptide variants were generated and tested for erythropoietin activities. Mutations were targeted to Epo-modification regions which include amino acids associated with carbohydrate content, aggregation, and EPOR binding properties of erythropoietin (see Example I). In addition, mutations outside of Epo-modification regions were generated. Several of the human erythropoietin polypeptide variants of the invention demonstrated increase or decreased activity in an erythroid precursor proliferation assay (see Example II). In addition, several of the erythropoietin polypeptide variants of the invention demonstrated a faster or slower time to reach a maximal level of erythroid precursor proliferation activity.

In one embodiment, the invention provides a human erythropoietin polypeptide variant containing a human erythropoietin amino acid sequence having an amino acid difference in two or more different Epo-modification regions and an enhanced erythropoietin activity, or a functional fragment thereof.

The invention additionally includes the use of synthetic polypeptides and peptidomimetics of the disclosed human erythropoietin polypeptide variants. Peptidomimetics include chemically modified peptides, peptide-like molecules containing non-naturally occurring amino acids, and peptoids, which are peptide-like molecules resulting from oligomeric assembly of N-substituted glycines. Peptidomimetics useful in the invention include, for example, peptide-like molecules which contain a constrained amino acid, a non-peptide component that mimics peptide secondary structure, or an amide bond isostere. See, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861.

The human erythropoietin polypeptide variants, or functional fragments thereof, of the invention can be generated using any of several methods known in the art. Methods for efficient synthesis and expression of populations of mutated polypeptides synthesized using oligonucleotide-directed mutagenesis can be performed, for example, as described in Wu et al., J. Mol. Biol., 294:151-162 (1999) and Kunkel, Proc. Natl. Acad. Sci. USA, 82:488-492 (1985) which are incorporated herein by reference. Also, for example, single or multiple amino acids mutations can be generated using oligonucleotides that code for the mutated amino acid(s) such as utilized in PCR based site-directed mutagenesis (for example, QuikChange™, Stratagene). Oligonucleotide-directed mutagenesis is a well known and efficient procedure for systematically introducing mutations, independent of their phenotype and is, therefore, suited for directed evolution approaches to protein engineering. The methodology is flexible, permitting precise mutations to be introduced without the use of restriction enzymes, and is relatively inexpensive.

Populations of changed polypeptides can also be generated using gene shuffling. Gene shuffling or DNA shuffling is a method that generates diversity by recombination as described, for example, in Stemmer, Proc. Natl. Acad. Sci. USA 91:10747-10751 (1994); Stemmer, Nature 370:389-391 (1994); Crameri et al., Nature 391:288-291 (1998); Stemmer et al., U.S. Pat. No. 5,830,721, which are incorporated herein by reference. Gene shuffling or DNA shuffling is a method using in vitro homologous recombination of pools of selected mutant genes. For example, a pool of point mutants of a particular gene can be used. The genes are randomly fragmented, for example, using DNase, and reassembled by PCR. If desired, DNA shuffling can be carried out using homologous genes from different organisms to generate diversity (Crameri et al., supra, 1998). The fragmentation and reassembly can be carried out, for example, in multiple rounds, if desired. The resulting reassembled genes are a population of variants that can be used in the invention.

Simultaneous incorporation of all of the encoding nucleic acids and all of the selected amino acid position changes can be accomplished by a variety of methods known to those skilled in the art, including for example, recombinant and chemical synthesis. Simultaneous incorporation can be accomplished by, for example, chemically synthesizing the nucleotide sequence for the region and incorporating at the positions selected for harboring variable amino acid residues a plurality of corresponding amino acid codons.

One method well known in the art for rapidly and efficiently producing a large number of alterations in a known amino acid sequence or for generating a diverse population of variable or random sequences is known as codon-based synthesis or mutagenesis. This method is the subject matter of U.S. Pat. Nos. 5,264,563 and 5,523,388 and is also described in Glaser et al. J. Immunology 149:3903 (1992), all of which are incorporated herein by reference. Variations to this synthesis method also exist and include, for example, the synthesis of predetermined codons at desired positions and the biased synthesis of a predetermined sequence at one or more codon positions. Other methods well known in the art for producing a large number of alterations in a known amino acid sequence or for generating a diverse population of variable or random sequences include, for example, degenerate or partially degenerate oligonucleotide synthesis. Codons specifying equal mixtures of all four nucleotide monomers, represented as NNN, results in degenerate synthesis. Whereas partially degenerate synthesis can be accomplished using, for example, the NNG/T codon.

The human erythropoietin polypeptide variants of the invention can be generated from corresponding nucleic acid molecules that encode the erythropoietin polypeptide variants. For example, the nucleic acid molecules disclosed herein as SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 217, 219, 221, 223, can be used to generate human erythropoietin polypeptide variants of the invention. This group of nucleic acid molecules will be referred to herein for convenience as SEQ ID NOS: 1-211 and 217-223 ODD. The nucleic acid molecules of the invention can be produced by any method of nucleic acid synthesis known to those skilled in the art. Such methods include, for example, chemical synthesis, recombinant synthesis, enzymatic polymerization and combinations thereof. These and other synthesis methods are well known to those skilled in the art.

For example, methods for synthesizing oligonucleotides can be found described in, for example, Oligonucleotide Synthesis: A Practical Approach, Gate, ed., IRL Press, Oxford (1984); Weiler et al., Anal. Biochem. 243:218 (1996); Maskos et al., Nucleic Acids Res. 20(7):1679 (1992); Atkinson et al., Solid-Phase Synthesis of Oligodeoxyribonucleotides by the Phosphitetriester Method, in Oligonucleotide Synthesis 35 (M. J. Gait ed., 1984); Blackburn and Gait (eds.), Nucleic Acids in Chemistry and Biology, Second Edition, New York: Oxford University Press (1996), and in Ansubel et al., Current Protocols in Molecular Biology, John Wiley and Sons, Baltimore, Md. (1999).

Recombinant and enzymatic synthesis, including polymerase chain reaction and other amplification methodologies can be found described in, for example, Sambrook et al., Molecular Cloning: A Laboratory Manual, Third Ed., Cold Spring Harbor Laboratory, New York (2001) and in Ansubel et al., (1999), supra. Solid-phase synthesis methods for generating arrays of oligonucleotides and other polymer sequences can be found described in, for example, Pirrung et al., U.S. Pat. No. 5,143,854 (see also PCT Application No. WO 90/15070), Fodor et al., PCT Application No. WO 92/10092; Fodor et al., Science (1991) 251:767-777, and Winkler et al., U.S. Pat No. 6,136,269; Southern et al. PCT Application No. WO 89/10977, and Blanchard PCT Application No. WO 98/41531. Such methods include synthesis and printing of arrays using micropins, photolithography and ink jet synthesis of oligonucleotide arrays.

Methods for synthesizing large nucleic acid polymers by sequential annealing of oligonucleotides can be found described in, for example, in PCT application No. WO 99/14318 and in U.S. Pat. No. 6,521,427, both to Evans and also described briefly below. Methods for automated synthesis and assembly similarly can be found described, for example, in the above patent publications as well as in WO 02/081490A2. These methods provide the ability to rapidly produce large polypeptide libraries with directed changes.

The design and computer synthesis of the human erythropoietin polypeptide variants can be performed by sequentially combining fragments of the complete gene, for example, in a 5′ to 3′ direction with overlapping oligonucleotides so that the complete gene is assembled. Assembly of the complete gene is accomplished by first electronically parsing the gene sequence into smaller oligonucleotide sequences (fragments) that can be more efficiently synthesized. The electronic parsing is performed for both the sense and complementary antisense strands of the full gene sequence. Parsing also is performed by maintaining partial complementarity between the 5′ terminus of either the sense or antisense strand and the 3′ terminus of its corresponding complementary sequence so that adjacent oligonucleotides can be annealed with a complementary oligonucleotide to form an overlapping oligonucleotide assembly for both strands that span the gene. The size of each parsed oligonucleotide can vary, but generally, will be between about 50-100 nucleotides (nt) in length with an about 50% overlap between complementary sense and antisense strands. For example, the oligonucleotides can be parsed using ParseOligo™, a proprietary computer program that optimizes nucleic acid sequence assembly. Optional steps in sequence assembly can include identifying and eliminating sequences that can give rise to hairpins, repeats or other difficult sequences. Additionally, the algorithm can first direct the synthesis of coding regions for each gene to correspond to a desired codon preference.

Following parsing into two sets of overlapping, partially complementary oligonucleotides, which represent the complete gene, the oligonucleotides are then synthesized. In this regard, the computer output of the parsed set of oligonucleotides for both the sense and antisense strand of the gene can be transferred to oligonucleotide synthesizer driver software. The synthesis of sequences of about 25 to 150 nt in length can be manufactured and assembled using the array synthesizer system and can be used without further purification. For example, two 96-well plates containing 100 nt oligonucleotides can yield a 9600 bp fragment of a gene cassette. Once synthesized, the individual oligonucleotides can be maintained in the original plates or transferred to new multi-well format plates for oligonucleotide assembly.

Assembly can be accomplished using, for example, robotics or microfluidics well known in the art for manipulating large numbers of oligonucleotide samples. Robotics and microfluidics allow synthesis and assembly to be performed rapidly and in a highly controlled manner. Such methods are described, for example, in WO 99/14318 and in U.S. application Ser. Nos. 60/262, 693 and 09/922, 221.

For example, oligonucleotide parsing from the genome sequence designed in the computer can be programmed for synthesis where sense and antistrands are placed in alternating wells of an array. Following synthesis in this format, the 12 row sequences of the gene are directed into a pooling manifold that systematically pools, for example, three wells into reaction vessels forming the triplex structure. Following temperature cycling for annealing and ligation, four sets of annealed triplex oligonucleotides are pooled into 2 sets of 6 oligonucleotide products, then 1 set of 12 oligonucleotide products. Each row of the synthetic array is associated with a similar manifold resulting in the first stage of assembly of 8 sets of assembled oligonucleotides representing 12 oligonucleotides each. The second manifold pooling stage is controlled by a single manifold that pools the 8 row assemblies into a single complete assembly. Passage of the oligonucleotide components through the two manifold assemblies (the first 8 and the second single) results in the complete assembly of all 96 oligonucleotides from the array. The assembly module of Genewriter™ can include a complete set of 7 pooling manifolds produced using microfabrication in a single plastic block that sits below the synthesis vessels. Various configurations of the pooling manifold will allow assembly of 96, 384 or 1536 well arrays of parsed component oligonucleotides. A similar strategy can be performed where pairs of oligonucleotides are pooled instead of triplets. The final complete gene can be generated using PCR with outside primers and the resulting product analyzed on an agarose gel.

Human erythropoietin polypeptide variants of the invention contain an amino acid difference in two or more different Epo-modification regions. The Epo-modification regions include internal amino acids associated with carbohydrate content, aggregation, and EPOR binding properties of erythropoietin. The inventors have found that the addition of a small number amino acids at the N-terminus of erythropoietin and the deletion of a small number of amino acids at the C-terminus of erythropoietin does not significantly change the erythroid precursor proliferation activity of the variant polypeptide. Other investigators have identified regions of erythropoietin that are associated with carbohydrate content, aggregation, and EPOR binding properties of erythropoietin.

A human erythropoietin polypeptide variant can have one amino acid difference in an Epo-modification region such as the aggregation region and one amino acid difference in a different Epo-modification region such as the EPOR binding region. In addition, a human erythropoietin polypeptide variant can have several amino acid difference in an Epo-modification region such as the aggregation region and one amino acid difference in a different Epo-modification region such as the EPOR binding region. Furthermore, a human erythropoietin polypeptide variant can have several amino acid difference in an Epo-modification region such as the aggregation region and several amino acid difference in a different Epo-modification region such as the EPOR binding region. Accordingly, a human erythropoietin polypeptide variant can contain, for example, from single to multiple amino acid differences in two or more Epo-modification regions.

Specific examples of human erythropoietin polypeptide variants containing one or more amino acid differences in two or more Epo-modification regions include SEQ ID NOS:6, 218, 220, 90, 222 and 224. These human erythropoietin polypeptide variants have been described above and also further below. For example, SEQ ID NO:90 contains one amino acid difference in an Epo-modification region corresponding to an aggregation region. This difference corresponds to the substitution of N to S at residue 24 (N24S) as set forth in FIG. 1. SEQ ID NO:90 also contains one or more amino acid differences in an Epo-modification region corresponding to an EPOR binding region. One difference corresponds to a change at residue 44 from T to S (T44S). Another difference corresponds to a change at residue 48 from F to M (F48M). Both T44S and F48M are located within an EPOR binding region. The human erythropoietin polypeptide variant set forth as SEQ ID NO:90 is encoded by the nucleic acid sequence shown in SEQ ID NO:89.

SEQ ID NOS:222 and 224 are related to SEQ ID NO:90 in that the three amino terminal residues set forth in FIG. 1 as residues −3 to −1 have been substituted with a mammalian leader sequence for directing extracellular secretion of the expressed human erythropoietin polypeptide variant. The leader sequence is encoded by the nucleotide sequence set forth in SEQ ID NO:225 and has the amino acid sequence set forth in SEQ ID NO:226, which is MGVHECPAWLWLLLSLLSLPLGLPVLG. Other mammalian or eukaryotic expression sequences well known in the art can substitute for this leader sequence. The polypeptide sequences of the unprocessed forms are shown in FIG. 2 for SEQ ID NOS:222 and 224. Other than the leader sequence replacement for mammalian extracellular expression, SEQ ID NO:222 is substantially identical to SEQ ID NO:90, in that it contains the amino acid differences in two or more different Epo-modification regions corresponding to N24S, T44S and F48M.

Compared to SEQ ID NO:222, SEQ ID NO:224 additionally contains amino acid differences compared to human erythropoietin at positions in an Epo-modification region directed to carbohydrate content. These amino acid differences in SEQ ID NO:224 correspond to P87V, W88N and P90T. Accordingly, SEQ ID NOS:90 and 222 contain, for example, three amino acid differences in two Epo-modification regions and SEQ ID NO:224 contains, for example, six amino acid differences in three Epo-modification regions. Following expression and processing in mammalian cells, the mature polypeptides of SEQ ID NOs:222 and 224 begin at residue 28, which corresponds to residue 1 (Ala) in FIG. 2. The human erythropoietin polypeptide variants set forth as SEQ ID NOS:222 and 224 are encoded by the nucleic acid sequences shown in SEQ ID NOS:221 and 223, respectively.

Human erythropoietin polypeptide variants containing one or more amino acid differences in two or more Epo-modification regions set forth as SEQ ID NOS:6, 218 and 220 are similarly related, for example, as variants with or without a mammalian leader sequence for extracellular expression. For example, SEQ ID NO:6 contains two amino acid differences in an Epo-modification region corresponding to an aggregation region. These differences correspond to substitutions at N24T and and N831 as set forth in FIG. 1. SEQ ID NO:6 also contains two acid differences in an Epo-modification region directed to carbohydrate content. One difference corresponds to a change at A30V. Another difference corresponds to a change at H32D. SEQ ID NO:6 additionally contains amino acid differences outside of an Epo-modification region. These differences correspond changes at W51R and V63I. The human erythropoietin polypeptide variant set forth as SEQ ID NO:6 is encoded by the nucleic acid sequence shown in SEQ ID NO:5.

SEQ ID NOS:218 and 220 are related to SEQ ID NO:6 by, for example, the replacement of the three amino terminal residues set forth in FIG. 1 as residues −3 to −1 with the leader sequence set forth as SEQ ID NO:226. The polypeptide sequences of the unprocessed forms of SEQ ID NOS:218 and 220 are shown in FIG. 2. Other than the leader sequence replacement, SEQ ID NO:218 is substantially identical to SEQ ID NO:6, in that it contains the above-described six amino acid differences in two or more different Epo-modification regions. Compared to SEQ ID NO:218, SEQ ID NO:220 contains the additional amino acid differences P87V, W88N and P90T for a total of nine amino acid changes compared to human erythropoietin. Following expression and processing in mammalian cells, the mature polypeptides of SEQ ID NOs:218 and 220 begin at residue 28, which corresponds to residue 1 (Ala) in FIG. 2. The human erythropoietin polypeptide variants set forth as SEQ ID NOS:218 and 220 are encoded by the nucleic acid sequences shown in SEQ ID NOS:217 and 219, respectively.

The carbohydrate portions of different glycoprotein molecules have been shown to have many diverse functions, including effects on the biosynthesis and secretion, immune protection, conformation, stability, solubility and biological activity of molecules (Skehel et al, Proc. Natl. Acad. Sci. USA 81:1779-1783 (1984); Cumming, D., Glycobiology 1:115-130 (1991)). For rHuEPO, in particular, it has been shown that the addition of carbohydrate is important for secretion from the cell, and for increasing the solubility of the molecule (Dube et al, J. Biol. Chem. 263:17516-17521 (1988); Narhi et al, J. Biol. Chem. 266:23022-23026 (1991); Delorme et al, Biochemistry 31:9871-9876 (1992)). Early research on erythropoietin from natural sources indicated that the sialic acid residues were important for biological activity in vivo (Lowry et al, Nature 185:102-103 (1960); Lukowsky and Painter, Can. J. Biochem. 50:909-917 (1972); Goldwasser et al, J. Biol. Chem. 249:4202-4206 (1974)). Removal of the sialic acid from either native erythropoietin or rHuEPO resulted in molecules having an increased activity in vitro, but low activity in vivo, presumably due to removal from circulation by the asialoglycoprotein receptor in the liver (Fukuda et al, Blood 73:84-89 (1989); Spivak and Hogans, Blood 73:90-99 (1989)). Similarly, it was shown that erythropoietin molecules, which have been deglycosylated to remove carbohydrate (or produced in E. coli to allow expression of only the non-glycosylated erythropoietin polypeptide), are active in vitro, but have low in vivo activity (Dordal et al, Endocrinology 116:2293-2299 (1985); Higuchi et al, J. Biol. Chem. 267:7703-7709 (1992)).

Carbohydrate addition (glycosylation) is a post-translational event that results in the addition of sugar chains to specific asparagine (N-linked) or serine/threonine (O-linked) amino acids in the polypeptide. The carbohydrate portion of natural and recombinant human erythropoietin consists of 3 N-linked sugar chains at Asn 24, 38 and 83, and one O-linked (mucin-type) sugar chain at Ser 126 (Browne et al, Cold Spring Harb. Symp. Quant. Biol. 51:693-702 (1986); Egrie et al, Immunobiology 172:213-224 (1986)).

In contrast to the invariant amino acid sequence of the protein portion of glycoproteins, the carbohydrate structures are variable, a feature referred to as microheterogeneity. For example, N-glycosylation sites on the same protein may contain different carbohydrate structures. Furthermore, even at the same glycosylation site on a given glycoprotein, different structures can be found. This heterogeneity is a consequence of the non-template directed synthesis of carbohydrates.

The carbohydrate structures of erythropoietin have been determined and the extent of the microheterogeneity defined for both rHuEPO and the natural hormone (Sasaki et al, J. Biol. Chem. 262:12059-12076 (1987); Sasaki et al., Biochemistry 27:8618-8626 (1988); Takeuchi et al, J. Biol. Chem. 263:3657-3663 (1988); Tsuda et al, Biochemistry 27:5646-5654 (1988)). An example of microheterogeneity for erythropoietin is seen on the N-linked carbohydrate chains, where the oligosaccharides can contain 2, 3 or 4 branches (or antennae), each of which is typically terminated with the negatively charged sugar molecule, sialic acid. With the exception of sialic acid, all of the other sugar molecules on erythropoietin are neutral. Similarly, the single O-linked carbohydrate can contain 0 to 2 sialic acid molecules. Since each of the 3 N-linked oligosaccharides can contain up to 4 sialic acid residues, and the single O-linked chain can contain 2, the erythropoietin molecule can have a maximum of 14 sialic acid residues. Therefore, because of the variability in sugar structure, the number of sialic acid molecules on erythropoietin varies, and as a consequence, so does the molecule's net negative charge. An isoform of erythropoietin is defined as a subset of the erythropoietin molecules that has a defined charge due to its sialic acid content. For reference, epoetin alfa (EPOGEN®, Amgen Inc, Thousand Oaks, Calif.), the source of the purified rHuEPO has been purified so as to contain isoforms 9-14.

It has been shown that a difference in biological activity can be attributed to the individual isoforms, with those isoforms having a higher sialic acid content exhibiting a progressively higher in vivo efficacy (Egrie and Brown, Brit. J. Cancer 84: (suppl.1) 3-10 (2001)). In addition, it was shown that the isoforms that have a higher sialic acid content have a higher in vivo biological activity, longer serum half-life and slower serum clearance. When a radioreceptor assay was used to determine binding of the various isoforms to the EPOR, it was found that the isoforms having a higher sialic acid content had a lower relative affinity for the EPO receptor. Taken together, these experiments indicate that the carbohydrate moieties of erythropoietin have an effect on the biological activity of the hormone, modulating both receptor affinity and serum clearance. There is a direct relationship between sialic acid content, in vivo biological activity, and serum half-life, but an inverse relationship with receptor affinity. Thus, modification of sialic acid content can alter erythropoietin properties.

Additional N-linked carbohydrate chains can be added to the rHuEPO molecule. N-linked carbohydrate is attached to the polypeptide backbone at a consensus sequence for carbohydrate addition (Asn-XXX-Ser/Thr). To introduce new carbohydrate attachment sites into the polypeptide backbone, the DNA sequence of the cloned human erythropoietin gene can be modified to code for one or more new consensus sequences. The consensus sequences are added at positions that are compatible with carbohydrate addition, for example, positions that do not interfere with receptor binding, or compromise the folding, conformation, or stability of the molecule. The erythropoietin analog, NESP, was generated by combining the carbohydrate addition sites of 2 successfully glycosylated 4-chain analogues into one molecule. The amino acid sequence of NESP differs from that of human erythropoietin at 5 positions (Ala30Asn, His32Thr, Pro87Val, Trp88Asn, and Pro90Thr) allowing for additional oligosaccharide attachment at asparagine residues at positions 30 and 88 (Elliott et al, Blood 96:82a (2000)).

Epo-modification regions also include regions that are known to be involved in erythropoietin polypeptide aggregation. Deglycosylated or Escherichia coli-derived non-glycosylated erythropoietin is prone to aggregation, making it difficult to carry out high-resolution structure analysis (Narhi et al., J. Biol. Chem. 266:23022-23026 (1991); Endo et al., J. Biochem. 112:700-706 (1992)). The isoelectric point of the non-glycosylated erythropoietin, determined by isoelectric focusing in the presence of urea, is about 9.2 (Davis et al., Biochemistry 26:2633-2638 (1987)). Mutation of the three Asn residues at positions 24, 38, and 83 at the N-linked glycosylation sites to Lys makes the isoelectric point more basic, resulting in an increase in the net positive charge at or below neutral pH and resulting in a decrease in the aggregation of the protein (Nahri et al., supra). Aggregation can be measured, for example, by monitoring the absorbance of a polypeptide solution at 287 nm. The refolded mutant erythropoietin was comparable to the human polypeptide in a receptor binding assay, yet showed a greater stability against heating and storage, with less aggregation. Thus, modification of amino acid residues at positions 24, 38, and 83 can alter erythropoietin properties.

Epo-modification regions also include regions that are known to be involved in erythropoietin binding to the erythropoietin receptor (EPOR). The EPOR is expressed on erythroid cells and also on embryonic stem cells, endothelial cells and neuronal cells. The structure of human erythropoietin complexed with EPOR shows that one molecule of erythropoietin binds two receptors (Syed et al., supra). Dimerization of the EPOR leads to activation of intercellular signaling pathways. Several allelic variations in the EPOR gene have been identified in different families. Some of these alleleic variations result in disease while others appear to have no effect.

Mutagenesis analysis has identified four amino acids in erythropoietin, T44, F48, N147, and L155 as being involved in binding to EPOR (Castelli et al., Pharm. Res. 41:313-318 (2000); Gareau et al., Nature 380:113 (1996); Lanse et al., Nature 405:635 (2000); Rich, I Molecular and Cellular Aspects of Erythropoietin and Erythropoiesis, Springer-Verlag, Berlin (1998); Syed et al., Nature 395:511-516 (1998); Middleton et al., J. Biol. Chem., 271:14045-14054 (1996); Middleton et al., J. Biol. Chem., 274:14163-14169 (1999). Binding of erythropoietin to its receptor can be determined using any of the receptor binding assay methods known in the art. For example, a radioreceptor assay (Broudy et al. Proc. Natl. Acad. Sci. USA 85:6513-6517 (1988)) can be used to measure the quantity of erythropoietin or erythropoietin polypeptide variant needed to displace 125I-rHuEPO bound to the EPOR on the surface of cells such as OCIM1 cells. The IC50, or amount of test compound required to compete 50% of the receptor-bound 125I-rHuEPO can be determined.

In addition to amino acid differences in two or more different Epo-modification domains, a human erythropoietin polypeptide variant of the invention can contain one or more amino acid differences outside of an Epo-modification domain. For example, the invention provides a human erythropoietin polypeptide variant containing a human erythropoietin amino acid sequence having an amino acid difference in two or more different Epo-modification regions, an amino acid difference located outside an Epo-modification region, and an enhanced erythropoietin activity, or a functional fragment thereof. Amino acid differences located outside an Epo-modification region can be located anywhere outside of the defined Epo-modification regions, for example, amino acid position 79 and amino acid position 146. A specific example of a human erythropoietin polypeptide variant of the invention with amino acid difference in two or more different Epo-modification regions and an amino acid difference in outside an Epo-modification region, with an enhanced erythropoietin activity is the polypeptide shown in SEQ ID NO:82 which contains amino acid differences at position 24 (aggregation region), position 97 (carbohydrate region) and position 79 (outside of Epo-modification region). Another example is SEQ ID NO:76 which contains amino acid differences at position 44 and 48 (EPOR region), position 83 (aggregation region), and position 79 and 146 (outside of Epo-modification region).

In a further embodiment, the invention provides a human erythropoietin polypeptide variant containing a human erythropoietin amino acid sequence having an amino acid difference in three Epo-modification regions, and an enhanced erythropoietin activity, or a functional fragment thereof. A specific example of a human erythropoietin polypeptide variant of the invention with amino acid difference in three Epo-modification regions and an enhanced erythropoietin activity is the polypeptide shown in SEQ ID NO:74 which contains amino acid differences at amino acid positions 30, 32, 87, and 88 (carbohydrate region), amino acid positions 38 and 83 (aggregation region), and amino acid position 155 (EPOR region).

The invention further provides a human erythropoietin polypeptide variant containing a human erythropoietin amino acid sequence having an amino acid difference in three Epo-modification regions, an amino acid difference located outside an Epo-modification region, and an enhanced erythropoietin activity, or a functional fragment thereof. In a related embodiment, the invention provides a human erythropoietin polypeptide variant containing a human erythropoietin amino acid sequence having an amino acid difference in three Epo-modification regions, an amino acid difference located outside an Epo-modification region, and an enhanced erythropoietin activity, or a functional fragment thereof, where an amino acid difference is located at position 146. As described above, the polypeptide shown in SEQ ID NO:74 is a specific example since it contains amino acid differences in all three Epo-modification regions and additionally contains amino acid differences at amino acid position 79 and 146.

The invention provides a human erythropoietin polypeptide variant, or functional fragment thereof, having an altered erythropoietin activity. For example, the human erythropoietin polypeptide variant can have enhanced or moderated activity compared to human erythropoietin.

As described further above, comparisons between human erythropoietin polypeptide variants and human erythropoietin can be determined using various types of human erythropoietin for comparison. For example, a source of human erythropoietin can be the World Health Organization Second International Reference Preparation. In addition, a source of human erythropoietin can be a commercially available form of human erythropoietin such as is available from Amgen. In addition, for example, a polypeptide variant with essentially identical activity as human erythropoietin can be used as a surrogate for human erythropoietin in subsequent assays. For example, as disclosed herein, the erythroid precursor proliferation activity of the polypeptide SEQ ID NO:2 is essentially identical to that of human erythropoietin obtained commercially from Amgen. Therefore, comparison of human erythropoietin polypeptide variants to the SEQ ID NO:2 polypeptide can be used to determine whether human erythropoietin polypeptide variant has altered activity compared to human erythropoietin.

The human erythropoietin polypeptide variants of the invention with altered erythropoietin activities can have different activities depending on the cell type that is being tested. For example, a human erythropoietin polypeptide variant of the invention can have a faster time to reach a maximal level of an erythropoietin activity in a T lymphocyte cell type and an slower time to reach a maximal level of same erythropoietin activity in an erythroid precursor cell type.

In addition, some human erythropoietin polypeptide variants of the invention may not show activity in one cell type, but can have activity in a different cell type. Furthermore, some erythropoietin polypeptide variants that do not show an activity in one cell type can show an activity in the same cell type if the time of the assay is extended. For example, a slow-acting erythropoietin polypeptide variant of the invention may not show any activity if an assay, such as an erythroid precursor proliferation assay, is monitored at 24 hours post-treatment, however an activity can be detected at 78 hours post-treatment. Therefore, erythropoietin polypeptide variants of the invention that do not appear to have activity can still have activity if tested in different cell types or under different conditions such as assay time length.

In one embodiment, the invention provides a human erythropoietin polypeptide variant containing a human erythropoietin amino acid sequence having an amino acid difference in two or more different Epo-modification regions and an enhanced erythropoietin activity, or a functional fragment thereof, where the enhanced erythropoietin activity includes increased erythroid precursor proliferation. For example, the invention provides a human erythropoietin polypeptide variant having an amino acid sequence selected from SEQ ID NOS: 6, 14, 18, 26, 28, 40, 48, 60, 62, 74, 76, 82, 90, 92, 94, 96, 100, 102, 104, 112, 132, 134, 138, 152, 160, 166, 184, 188, 192, 206, 212, 218, 220, 222, 224 or a functional fragment thereof.

A human erythropoietin polypeptide variant having increased erythroid precursor proliferation activity can be useful in the treatment of individuals who have a disease such as anemia, chronic renal failure, cancer or a neurological condition. In one embodiment, a composition containing a human erythropoietin polypeptide variant of the invention having increased erythroid precursor proliferation activity is used to treat an individual who does not respond, or who responds sub-optimally to therapy with available erythropoietin polypeptides such as rHuEPO or NESP.

Erythroid precursor proliferation and erythroid cell viability can be determined using several assays that are known in the art. For example, methods of measuring cell proliferation include the detection of proliferation associated antigens by immunohistochemistry, quantitation of DNA synthesis by measuring tritiated thymidine or bromodeoxyuridine uptake, and quantitation of reduction of the intracellular environment by tetrazolium salt reduction. For example, an in vitro bioassay that measures tritiated thymidine uptake in an erythropoietin dependent cell line such as 32D+EPOR can be used in the invention (Pacifici and Thomason, J. Biol. Chem. 269:1571 (1994)).

The invention provides a method useful for rapidly screening several erythropoietin polypeptide variants in real-time. For example, the invention provides a method of measuring erythroid precursor proliferation activity of an erythropoietin polypeptide over time, by: (a) contacting an erythroid precursor cell line with a erythropoietin polypeptide and a non-toxic proliferation-sensitive dye under conditions sufficient for cell viability, and (b) measuring a signal of the dye at various times after the contacting, where changes in the signal of the dye correlate with erythroid precursor proliferation activity. The invention further provides a method of measuring erythroid precursor proliferation activity of a human erythropoietin polypeptide variant over time, by: (a) contacting an erythroid precursor cell line with a human erythropoietin polypeptide variant and a non-toxic proliferation-sensitive dye under conditions sufficient for cell viability, and (b) measuring a signal of the dye at various times after the contacting, where changes in the signal of the dye correlate with erythroid precursor proliferation activity.

As described herein the UT-7 cell line is an example of an erythroid precursor cell line that can be used in the methods of the invention (Example II). An example of a non-toxic proliferation-sensitive dye is the alamarBlue™ (BioSource) dye reagent which can be used to measure the intracellular environment. The internal environment of proliferating cells is known to be more reduced than that of non-proliferating cells and this reduced state can be measured using alamarBlue™. Specifically the ratios of NADPH/NADP, FADH/FAD, FMNH/FMN and NADH/NAD increase during proliferation. AlamarBlue™ is a soluble, non-toxic stable dye that changes color in response to oxidation and reduction. Because alamarBlue™ is non-toxic, an advantage to this method is that continuous monitoring of cells in culture is permitted. Non-toxicity is advantageous when assaying variant polypeptides because variants with activity that may not be detected in a single time point assay can be detected when measurements can be made in real-time. Another advantage is that a single well containing the variant polypeptide can be assayed several times which reduces the amount of reagents that are required compared to setting up several wells with different time points. In addition, a single well assay as described herein has reduced variability compared to multiple well assays.

A signal from the dye can be measured at various time points, for example, by measuring the absorbance of the dye at various time points (Example II). For example, proliferation measurements with alamarBlue™ can be made either spectrophotometrically by monitoring the absorption of alamarBlue™ supplemented cell culture media at two wavelengths, or alternatively, proliferation measurement with alamarBlue™ can be made fluorometrically.

The assays described above for determining erythroid precursor proliferation can be used as a an initial screen to test for activity or can be used to more precisely determine activity. For example, a pre-determined volume of a supernatant containing a human erythropoietin polypeptide variant can be tested in such an assay as an initial screen to find variants with enhanced activity. A further assay can then be performed using a pre-determined quantity of polypeptide variant to obtain more precise comparisons to human erythropoietin or to other variants. The quantity of erythropoietin polypeptide present in a sample such as a supernatant can be determined using several assays well known in the art. For example, an immunoassay, such as an ELISA, utilizing an antibody or antibodies that react with human erythropoietin can be used to quantitate the level of human erythropoietin polypeptide or polypeptide variant in a sample. Several antibodies are known in the art that can be used in immunoassays such as RIA-P, RIA-N, E1A/P11, E1A/F13 and RIA-9G8A (Elliott et al., Blood 87:2702-2713 (1996))). In order to detect the quantity of a variant polypeptide, more than one antibody can be used in case the variant has changes in an epitope recognized by one of the antibodies.

As disclosed herein, several of the human erythropoietin polypeptide variants disclosed herein show increased erythroid precursor proliferation activity in an alamarBlue™ based assay using the UT-7 cell line (Example II and Table I). For example, the polypeptides shown as SEQ ID NOS:90, 222, 224 have increased erythroid precursor proliferation activity.

In addition to the UT-7 cell line, erythropoietin activity can also be measured, for example, using the human erythroleukaemic cell line TF-1. Erythropoietin activity can also be measured using phenylhydrazin-treated mouse spleen cells as a source of erythroid precursor cells. Furthermore, bone marrow colony assays can be used to detect erythropoietin either by monitoring colonies or burst-forming units of erythrocytes in methylcellulose cultures.

In one embodiment, the invention provides a human erythropoietin polypeptide variant containing a human erythropoietin amino acid sequence having an amino acid difference in two or more different Epo-modification regions and an enhanced erythropoietin activity, or a functional fragment thereof, where the enhanced erythropoietin activity includes increased erythroid precursor differentiation. Methods for determining erythroid precursor differentiation are known in the art and include, for example, microscopic techniques which identify morphological changes that accompany differentiation and antibody staining techniques such as FACS which identify cell surface markers associated with differentiated cells. In addition, erythroid precursor differentiation can be assessed by hemoglobin formation assayed by dianisidine staining and subsequent cell counting.

In addition to in vitro assays used to determine erythroid precursor proliferation and differentiation, measurements of these processes can be determined indirectly in vivo, for example, by a hematocrit or hemoglobin measurement from a blood sample as described herein.

A hematocrit is described as the percent of whole blood that is comprised of red blood cells (RBCs). Hematocrit is also known by the terms erythrocyte count or red blood cell count. The hematocrit is a compound measure of RBC number and size. The test is performed from blood drawn from a vein of an individual. The blood is centrifuges and the cellular portion is compared to the total amount of blood and expressed as a percent. The cellular portion is almost entirely red blood cells, the percent that is white blood cells is small. Normal hematocrit values depend on the gender of the individual and health status of the individual. For example, hematocrit can be influenced by dehydration and dialysis. A normal value can be, for example, males: 40% to 54% of total blood volume made up of RBCs, females: 37% to 47% of total blood volume made up of RBCs, and dialysis patients: 30% to 36% of total blood volume made up of red blood cells.

In another embodiment, the invention provides a human erythropoietin polypeptide variant containing a human erythropoietin amino acid sequence having an amino acid difference in two or more different Epo-modification regions and an enhanced erythropoietin activity, or a functional fragment thereof, where the enhanced erythropoietin activity includes a faster time to reach a maximal level of an erythropoietin activity. For example, the invention provides a human erythropoietin polypeptide variant containing a human erythropoietin amino acid sequence having an amino acid difference in two or more different Epo-modification regions and an enhanced erythropoietin activity, or a functional fragment thereof, where the enhanced erythropoietin activity includes a faster time to reach a maximal level of erythroid precursor proliferation. For example, the invention provides a human erythropoietin polypeptide variant having an amino acid sequence selected from SEQ ID NOS: 26, 70, 96, 112, 138, 152, or a functional fragment thereof.

The time to reach a maximal level of an erythroid activity, such as erythroid precursor proliferation, can be determined using several assays that are known in the art. For example, any of the assays described above for determining the level of activity of erythroid precursor proliferation can be used to determine the time to reach a maximal level of proliferation activity. For example, the time to reach a maximal level of erythroid proliferation activity was measured in Example II and Table 1 using the alamarblue™ assay described above. In this assay, the time to reach a maximal level of erythroid proliferation activity of erythropoietin variant polypeptides is compared to the time to reach a maximal level of activity by the polypeptide encoded by SEQ ID NO:2. Since the polypeptide encoded by SEQ ID NO:2 had essentially identical activity when compared to human erythropoietin SEQ ID NO:214, the polypeptide encoded by SEQ ID NO:2 was used as a surrogate for human erythropoietin.

Several temporal aspects of an erythropoietin activity can be utilized in the invention. For example, in addition to the time it takes to reach a maximal level of an erythropoietin activity, the initial time of onset of an erythropoietin activity can be measured. For example, a human erythropoietin polypeptide variant of the invention can have a faster or slower time of onset of an activity such as onset of erythroid precursor proliferation.

In one embodiment, a composition containing a human erythropoietin polypeptide variant of the invention having a faster time to reach a maximal level of erythroid precursor proliferation is used to treat an individual who does not respond, or who responds sub-optimally to therapy with available erythropoietin polypeptides such as rHuEPO or NESP. In addition, because the therapeutic effects of treatment with available erythropoietin polypeptides such as rHuEPO or NESP can take several days, a faster-acting erythropoietin polypeptide variant can be used to get therapeutic results more rapidly. A more rapid result can be useful in an individual, for example, who has critically low hematocrit or hemaglobin levels.

As described above, a human erythropoietin polypeptide variant of the invention can have an altered erythropoietin activity compared to human erythropoietin shown as SEQ ID NO:214. Different properties of an erythropoietin polypeptide can affect activity in vitro or in vivo. Enhanced activity can be due to a change in any property of the polypeptide such as increased binding to receptor, increased plasma stability, or changes in the pharmacological properties of an erythropoietin polypeptide such as absorption, distribution, metabolism and/or elimination (ADME) of the polypeptide. Altered properties in a human erythropoietin polypeptide variant can result in increased or decreased levels of activity, an different side-effect profile, a different time of onset or time to reach a maximal activity, differences in the ability of a patient to tolerate the polypeptide, and increased circulating half-life of an erythropoietin polypeptide variant.

Properties associated with erythropoietin polypeptide variants such as in vivo half-life and ADME properties can be determined using methods well known in the art. For example, in vivo half-life of an erythropoietin polypeptide can be determined using New Zealand while rabbits that are injected intravenously with specified amounts of an erythropoietin polypeptide of interest. Blood samples are then obtained at specified times and serum prepared. Serum erythropoietin levels are then determined using an in vitro bioasssay, for example, an alamarBlue™ assay as described herein and compared to human erythropoietin.

In addition to providing human erythropoietin polypeptide variants with enhanced activity, the invention likewise provides human erythropoietin polypeptide variants with moderated activity. In one embodiment, the invention provides a human erythropoietin polypeptide variant containing a human erythropoietin amino acid sequence having an amino acid difference in two or more different Epo-modification regions and a moderated erythropoietin activity, or a functional fragment thereof.

In addition to amino acid differences in two or more different Epo-modification domains, a human erythropoietin polypeptide variant of the invention can contain one or more amino acid differences outside of an Epo-modification domain. For example, the invention provides a human erythropoietin polypeptide variant containing a human erythropoietin amino acid sequence having an amino acid difference in two or more different Epo-modification regions, an amino acid difference located outside an Epo-modification region, and a moderated erythropoietin activity, or a functional fragment thereof. Amino acid differences located outside an Epo-modification region can be located anywhere outside of the defined Epo-modification regions, for example, amino acid position 79 and amino acid position 146. A specific example of a human erythropoietin polypeptide variant of the invention with amino acid difference in two or more different Epo-modification regions and an amino acid difference in outside an Epo-modification region, with a moderated erythropoietin activity is the polypeptide shown in SEQ ID NO: 174 which contains amino acid differences at position 38 (aggregation region), positions 44 and 48 (EPOR region), position 79, 146, and 154 (outside of Epo-modification region).

In a further embodiment, the invention provides a human erythropoietin polypeptide variant containing a human erythropoietin amino acid sequence having an amino acid difference in three Epo-modification regions, and a moderated erythropoietin activity, or a functional fragment thereof. A specific example of a human erythropoietin polypeptide variant of the invention with amino acid difference in three Epo-modification regions and a moderated erythropoietin activity is the polypeptide shown in SEQ ID NO: 198 which contains amino acid differences at amino acid position 32 (carbohydrate region), amino acid position 38 (aggregation region), and amino acid position 155 (EPOR region).

The invention further provides a human erythropoietin polypeptide variant containing a human erythropoietin amino acid sequence having an amino acid difference in three Epo-modification regions, an amino acid difference located outside an Epo-modification region, and a moderated erythropoietin activity, or a functional fragment thereof. As described above, the polypeptide shown in SEQ ID NO: 198 is a specific example since it contains amino acid differences in all three Epo-modification regions and additionally contains amino acid differences at amino acid position 17, 51, 79, and 98. In a related embodiment, the invention provides a human erythropoietin polypeptide variant containing a human erythropoietin amino acid sequence having an amino acid difference in three Epo-modification regions, an amino acid difference located outside an Epo-modification region, and a moderated erythropoietin activity, or a functional fragment thereof, where an amino acid difference is located at position 146. For example, the polypeptide shown in SEQ ID NO:68 contains an amino acid difference at positions 24 and 38 (aggregation region), position 87 (carbohydrate region), position 44 (EPOR region) and amino acid positions 79 and 146 (outside of Epo-modification region).

In one embodiment, the invention provides a human erythropoietin polypeptide variant containing a human erythropoietin amino acid sequence having an amino acid difference in two or more different Epo-modification regions and a moderated erythropoietin activity, or a functional fragment thereof, where the moderated erythropoietin activity includes decreased erythroid precursor proliferation. For example, the invention provides a human erythropoietin polypeptide variant having an amino acid sequence selected from SEQ ID NOS: 4, 8, 68, 70, 150, 170, 174, 198, or a functional fragment thereof. In one embodiment, a composition containing a human erythropoietin polypeptide variant of the invention having decreased erythroid precursor proliferation activity is used to treat an individual who, for example, responds to therapy with available erythropoietin polypeptides such as rHuEPO or NESP with a drastic increase in hematocrit.

In another embodiment, the invention provides a human erythropoietin polypeptide variant containing a human erythropoietin amino acid sequence having an amino acid difference in two or more different Epo-modification regions and a moderated erythropoietin activity, or a functional fragment thereof, where the moderated erythropoietin activity includes decreased erythroid precursor differentiation.

In a further embodiment, the invention provides a human erythropoietin polypeptide variant containing a human erythropoietin amino acid sequence having an amino acid difference in two or more different Epo-modification regions and a moderated erythropoietin activity, or a functional fragment thereof, where the moderated erythropoietin activity includes a slower time to reach a maximal level of an erythropoietin activity. For example, the invention provides a human erythropoietin polypeptide variant containing a human erythropoietin amino acid sequence having an amino acid difference in two or more different Epo-modification regions and a moderated erythropoietin activity, or a functional fragment thereof, where the moderated erythropoietin activity includes a slower time to reach a maximal level of erythroid precursor proliferation. For example, the invention provides a human erythropoietin polypeptide variant having an amino acid sequence selected from SEQ ID NOS: 8, 14, 28, 40, 48, 60, 62, 68, 74, 90, 94, 100, 104, 132, 150, 166, 174, 184, 206, 212, or a functional fragment thereof.

In one embodiment, a composition containing a human erythropoietin polypeptide variant of the invention having a slower time to reach a maximal level of activity or having a slower time of onset of an activity is used to treat an individual who, for example, responds to therapy with available erythropoietin polypeptides such as rHuEPO or NESP with a drastic increase in hematocrit. In this case, a slower-acting erythropoietin polypeptide variant can result in a more gradual increase in hematocrit. In addition, a slower-acting erythropoietin polypeptide variant can give a more even or sustained result in the same way that a time-release formulation of a drug can result in a more even or sustained result.

The invention also provides an erythropoietin polypeptide variant having an amino acid sequence selected from SEQ ID NOS: 2-212 and 218-224 EVEN, or functional fragment thereof. The invention further provides an erythropoietin polypeptide variant having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, or functional fragment thereof.

Other human erythropoietin polypeptide variants useful in the invention are human erythropoietin polypeptide variants, or functional fragment thereof, where the human erythropoietin polypeptide variant has substantially the same amino acid sequence as referenced in SEQ ID NOS: 2-212 and 218-224 EVEN. Substantially the same amino acid sequence is intended to mean an amino acid sequence contains a considerable degree of sequence identity or similarity, such as at least 70%, 80%, 90%, 95%, 98%, or 100% sequence identity or similarity, to a reference amino acid sequence. Substantially the same amino acid sequence includes conservative and non-conservative amino acid changes, gaps, and insertions to an amino acid sequence. Conservative and non-conservative amino acid changes, gaps, and insertions to an amino acid sequence can be compared to a reference sequence using available algorithms and programs such as the Smith-Waterman algorithm and the BLAST homology search program (Altschul et al., J. Mol. Biol. 215:403-410 (1990)).

Various modifications of a human erythropoietin polypeptide variant primary amino acid sequence can result in polypeptides having substantially equivalent functions as compared to the unmodified sequence. Those skilled in the art can determine which residues and which regions of a human erythropoietin polypeptide variant sequence are likely to be tolerant of modification and still retain an activity associated with the unmodified sequence. For example, amino acid substitutions or chemical or enzymatic modifications at residues that are less well conserved between species are more likely to be tolerated than substitutions at highly conserved residues. Accordingly, an alignment can be performed among erythropoietin sequences of various species to determine residues and regions in which modifications are likely to be tolerated. Additional guidance for determining residues and regions of erythropoietin likely to be tolerant of modification is provided by studies of erythropoietin fragments and variants.

The invention also provides an isolated nucleic acid molecule containing a nucleic acid sequence encoding a human erythropoietin polypeptide variant having an amino acid sequence selected from the group consisting of SEQ ID NOS: 2-212 and 218-224 EVEN, or a functional fragment thereof. The invention further provides an isolated nucleic acid molecule containing a nucleic acid sequence encoding a human erythropoietin polypeptide variant having an amino acid sequence selected from the group consisting of SEQ ID NOS: 4, 6, 8, 14, 16, 18, 26, 28, 30, 34, 36, 385, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 224, or a functional fragment thereof. The invention also provides an isolated nucleic acid molecule containing a nucleic acid sequence having a nucleic acid sequence selected from the group consisting of SEQ ID NOS: 1-211 and 217-223 ODD, or a functional fragment thereof.

Other compositions useful in the invention are isolated nucleic acid molecules containing a nucleic acid sequence having substantially the same nucleic acid sequence as a nucleic acid sequence selected from the group consisting of SEQ ID NOS: 1-211 and 217-223 ODD, or a functional fragment thereof. Substantially the same nucleic acid sequence is intended to mean a nucleic acid sequence contains a considerable degree of sequence identity or similarity, such as at least 70%, 80%, 90%, 95%, 98%, or 100% sequence identity or similarity, to a reference nucleic acid sequence. Substantially the same nucleic acid sequence can include gaps, and insertions to a nucleic acid sequence. Modifications to a nucleic acid sequence can be compared to a reference sequence using available algorithms and programs such as the Smith-Waterman algorithm and the BLAST homology search program (Altschul et al., J. Mol. Biol. 215:403-410 (1990)).

Various modifications of a human erythropoietin polypeptide variant nucleic acid sequence can result in nucleic acid molecules having substantially equivalent functions as compared to the unmodified sequence. Those skilled in the art can determine which nucleotides and which regions of a human erythropoietin nucleic acid sequence are likely to be tolerant of modification and still retain an activity associated with the unmodified sequence.

In one embodiment the invention provides a vector that contains an isolated nucleic acid molecule containing a nucleic acid sequence encoding a human erythropoietin polypeptide variant having an amino acid sequence selected from the group consisting of SEQ ID NOS: 2-212 and 218-224 EVEN, or a functional fragment thereof. In another embodiment the invention provides a host cells containing this vector.

The invention further provides a method of producing a human erythropoietin polypeptide variant by growing the host cell described above under conditions sufficient for expression of the erythropoietin polypeptide variant. The invention further provides a method of producing a human erythropoietin polypeptide variant by growing the host cell described above under conditions sufficient for expression of the erythropoietin polypeptide variant, and isolating the erythropoietin polypeptide variant from the host cell or host cell medium.

Vectors useful in the invention include autonomously replicating recombinant expression constructs such as plasmid and viral DNA vectors. The invention includes vectors where erythropoietin polypeptide variant-encoding nucleic acids are operatively linked to an endogenous or exogenous promoter, enhancer, or operator sequence and a transcription terminator sequence. Promoter and enhancer sequences are generally selected for the ability to increase gene expression, while operator sequences are generally selected for the ability to regulate gene expression. It is understood in the art that the choice of host cell is relevant to selection of an appropriate regulatory sequence. Vectors used in the invention can also include sequences encoding one or more selectable markers that permit identification of host cells bearing the construct. Vectors can also include sequences that facilitate homologous recombination in a host cell.

Suitable vectors for expression in prokaryotic or eukaryotic cells are well known to those skilled in the art (see, for example, Ausubel et al., supra, 1999). Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. A vector can include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art. One skilled in the art will know or can readily determine an appropriate promoter for expression in a particular host cell.

Vectors useful for expression of a human erythropoietin polypeptide variant can contain a regulatory element that provides tissue specific or inducible expression of an operatively linked nucleic acid. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.)); metallothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammary tumor virus (MMTV) induced by steroids such as glucocortocoid and estrogen (Lee et al., Nature, 294:228-232 (1981); and heat shock promoters inducible by temperature changes.

In addition, viral vectors such as retroviral, adenovirus, adeno-associated virus, lentivirus, and herpesvirus vectors can be used to express ATX polypeptides into a cell. Viral based systems provide the advantage of being able to introduce relatively high levels of a heterologous nucleic acid into a variety of cells. Additionally, such viruses can introduce heterologous DNA into nondividing cells. Viral vectors include, for example, Herpes simplex virus vectors (U.S. Pat. No. 5,501,979), Vaccinia virus vectors (U.S. Pat. No. 5,506,138), Cytomegalovirus vectors (U.S. Pat. No. 5,561,063), Modified Moloney inurine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

The invention further provides a host cell containing a human erythropoietin polypeptide variant-encoding vector as described above. For example, the invention provides a host cell that contains a vector which contains an isolated nucleic acid molecule having the same or substantially the same nucleotide sequence as SEQ ID NO: 1-211 and 217-223 ODD. Host cells include prokaryotic and eukaryotic cells. Nucleic acids of the invention can be introduced into the host cell as part of a circular plasmid, or as linear DNA having an isolated protein coding region or a viral vector. Methods for introducing DNA into the host cell are well known in the art and include transformation, transfection, electroporation, nuclear injection, or fusion with carriers such as liposomes, micelles, ghost cells, protoplasts, and other transformed cells. Detailed procedures for these methods can be found in Sambrook et al., Molecular Cloning: A Laboratory Manual (Cold Spring Harbor Laboratory Press, 1989) and the references cited therein). Expression systems of the invention include bacterial, yeast, fungal, plant, insect, invertebrate, and mammalian cells systems.

Useful mammalian expression vectors and methods of introducing such vectors into mammalian cells either ex vivo or in vivo, for expression of the encoded polypeptide, are well known in the art. For example, a plasmid expression vector can be introduced into a cell by calcium-phosphate mediated transfection, DEAE-Dextran-mediated transfection, lipofection, polybrene- or polylysine-mediated transfection, electroporation, or by conjugation to an antibody, gramacidin S, artificial viral envelopes or other intracellular carriers. A viral expression vector can be introduced into a cell in an expressible form by infection or transduction, for example, or by encapsulation in a liposome.

In one embodiment, the invention provides a method of producing a human erythropoietin polypeptide variant by growing a host cell that contains a human erythropoietin polypeptide variant vector under conditions sufficient for expression of the erythropoietin polypeptide variant. In a further embodiment, the erythropoietin polypeptide variant can be isolated from the host cell or the host cell medium using methods described below and known in the art.

Erythropoietin polypeptide variants can be isolated from the cells, or from the medium in which the cells are grown, by purification methods known in the art, for example, conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those wherein the desired protein is expressed and purified as a fusion protein having a specific tag, label, or chelating moiety that is recognized by a specific binding partner or agent. The purified protein can be cleaved to yield the desired protein, or be left as an intact fusion protein. A description of the purification of recombinant erythropoietin from cell medium that supported the growth of mammalian cells containing recombinant erythropoietin plasmids for example, is included in U.S. Pat. No. 4,667,016 to Lai et al., which is incorporated herein by reference.

The nucleic acid composition of the invention can be used in gene therapy methods to restore erythropoietin activity thus treating those disease states described herein. Delivery of a functional erythropoietin polypeptide variant gene to appropriate cells can be effected ex vivo, in situ, or in vivo by use of vectors, for example, viral vectors such as adenovirus, adeno-associated virus, or a retrovirus, or ex vivo by use of physical DNA transfer methods such as liposomes or chemical treatments (Anderson, Nature, supplement to vol. 392, no. 6679, pp.25-20 (1998)). Alternatively, in some human disease states, preventing the expression of, or inhibiting the activity of, erythropoietin can be useful in treating the disease states. In this case, anti-sense therapy or gene therapy, for example, where a dominant negative erythropoietin mutant is introduced into a target cell type, can be applied to negatively regulate the expression of erythropoietin. In addition, interferring RNAs derived from compositions of the invention can be used to inhibit the activity of erythropoietin (Brunumelkamp et al., Science 296:550-553 (2002); Elbashir et al., Nature 411:494-498 (2002)). RNAi is a process of sequence-specific gene silencing by post-transcriptional RNA degradation, which is initiated by double-stranded RNA (dsRNA) homologous in sequence to the silenced gene.

The invention provides an antibody, or functional binding fragment thereof, containing a region that specifically binds to a human erythropoietin polypeptide variant containing an amino acid sequence as shown in SEQ ID NOS: 2-212 and 218-224 EVEN. The invention further provides an antibody, or functional binding fragment thereof, which specifically binds to a human erythropoietin polypeptide variant containing an amino acid sequence as referenced in SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, -148, 150, 152, 160, 162, 164, 166, 170, 172, 174, 184, 188, 192, 194, 196, 198, 200, 206, 210, 212, 218, 220, 222, 224.

The term specifically binds is intended to mean the molecule will have an affinity for the target molecule that is measurably higher than its affinity for a non-specific interaction. For example, an antibody that specifically binds another polypeptide will have an affinity for the target polypeptide or antigen that is measurably higher than its affinity for a non-specific interaction. Binding affinity can be low or high affinity so long as the binding is sufficient to be detectable. For example, an antibody can bind a human erythropoietin polypeptide variant with a binding affinity (Kd) of about 10⁻⁵ M or less, 10⁻⁶ M or less, about 10⁻⁷ M or less, including about 10⁻⁸ M or less, such as 10⁻⁹ M or less. Several methods for detecting antibody binding are well known in art.

Antibodies of the invention include, for example, monoclonal and polyclonal antibodies, single chain antibodies, chimeric antibodies, bifunctional or bispecific antibodies, humanized antibodies, human antibodies, and complementary determining region (CDR)-grafted antibodies, including compounds which include CDR or antigen-binding sequences, which specifically bind to a polypeptide of the invention. Antibody fragments, including Fab, Fab′, F(ab′)₂, and Fv, are also provided by the invention. Screening assays to determine binding specificity or exclusivity of an antibody of the invention are well known in the art (see Harlow et al. (Eds), Antibodies A Laboratory Manual; Cold Spring Harbor Laboratory; Cold Spring Harbor, N.Y. (1988)).

Antibodies of the invention can be produced using any method well known in the art, using any polypeptide, or immunogenic fragment thereof, of the invention. Immunogenic polypeptides can be isolated from natural sources, from recombinant host cells, or can be chemically synthesized. Polypeptide of the invention can also be conjugated to a hapten such as keyhole limpet hemocyanin (KLH) in order to increase immunogenicity. Methods for synthesizing such peptides are known in the art, for example, as in R. P. Merrifield, J. Amer. Chem. Soc. 85: 2149-2154 (1963); J. L. Krstenansky, et al., FEBS Lett. 211:10 (1987). Antibodies to a polypeptide of the invention can also be prepared through immunization using a nucleic acid of the invention, as described in Fan et al., Nat. Biotech. 17:870-872 (1999). DNA encoding a polypeptide can be used to generate antibodies against the encoded polypetide following topical administration of naked plasmid DNA or following injection, for example, intramuscular injection, of the DNA.

Non-human antibodies can be humanized by any methods known in the art. In one method, the non-human CDRs are inserted into a human antibody or consensus antibody framework sequence. Further changes can then be introduced into the antibody framework to modulate affinity or immunogenicity. Antibodies of the invention further include plastic antibodies or molecularly imprinted polymers (MIPs) (Haupt and Mosbauch, TIBTech 16:468-475 (1998)). Antibodies of this type can be useful in immunoaffinity separation, chromatography, solid phase extraction, immunoassays, for use as immunosensors, and for screening chemical or biological libraries. Advantages of antibodies of this type are that no animal immunization is required, the antibodies are relatively inexpensive to produce, they are resistant to organic solvents, and they are reusable over long period of time.

Antibodies that bind to erythropoietin polypeptide variants of the invention can be used in diagnostic and therapeutic methods. For example, an antibody of the invention can be labeled with a detection moiety and used to detect the presence, absence or amount of an erythropoietin polypeptide of the invention in vivo, in vitro, or in situ. In addition, an antibody of the invention can be labeled with a therapeutic moiety such as chemotherapeutic agent and used, for example, to reduce the number of cells that can internalize these polypeptides. Further, an antibody of the invention can act in a competitive or dominant negative fashion to interfere with or reduce an erythropoietin activity.

A moiety, such as a fluorescent molecule, can be linked to a polypeptide, including an antibody, of the invention at any location within the polypeptide. Chemistries used for the linkage of various moieties to polypeptides are well known in the art. A moiety such as detection moiety can be linked to a polypeptide, including an antibody, of the invention using, for example, carbodiimide conjugation (Bauminger and Wilchek, Meth. Enzymol. 70:151-159 (1980)). Carbodiimides comprise a group of compounds that have the general formula R—N═C═N—R′, where R and R′ can be aliphatic or aromatic, and are used for synthesis of polypeptide bonds. The preparative procedure is simple, relatively fast, and is carried out under mild conditions. Carbodiimide compounds attack carboxylic groups to change them into reactive sites for free amino groups. Carbodiimide conjugation has been used to conjugate a variety of compounds to carriers for the production of antibodies. The water soluble carbodiimide, 1-ethyl-3-(3-dimethylaminopropyl) carbodiimide (EDC) is useful for conjugating a moiety to a polypeptide, including an antibody of the invention.

The invention provides a composition containing a human erythropoietin polypeptide variant containing an amino acid sequence selected from SEQ ID NOS: 2-212 and 218-224 EVEN and a pharmaceutically acceptable medium. The invention further provides a composition containing a human erythropoietin polypeptide variant containing an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, and a pharmaceutically acceptable medium. For example, the composition can be a pharmaceutical composition comprising an effective amount of a human erythropoietin polypeptide variant and a pharmaceutically acceptable diluent, adjuvant or carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain a physiologically acceptable compound that acts, for example, to stabilize or to increase the absorption of the composition. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the composition. If desired, the pharmaceutical composition also can contain a second active agent such as a therapeutic agent.

One skilled in the art understands that a composition containing a human erythropoietin polypeptide variant of the invention can be administered to an individual by various routes including, for example, orally or parenterally, such as intravenously, or by injection or intubation. In performing a therapeutic method as disclosed herein, an effective amount of the composition must be administered to the subject. An effective amount is the amount of the conjugate that produces a desired effect and depends, for example, on the particular disease that is being treated.

The route of administration of the compositions of the invention will depend, in part, on the chemical structure of the erythropoietin polypeptide variants. Some polypeptides, for example, are not particularly useful when administered orally because they can be degraded in the digestive tract. However, methods for chemically modifying a polypeptide to render it less susceptible to degradation by endogenous proteases or more absorbable through the alimentary tract are well known in the art (see, for example, Blondelle et al., supra, 1995; Ecker and Crooke, supra, 1995; Goodman and Ro, supra, 1995). In addition, methods for preparing libraries of polypeptide analogs such as polypeptides containing D-amino acids; peptidomimetics consisting of organic molecules that mimic the structure of a polypeptide; or peptoids such as vinylogous peptoids, have been previously described above and can be used to identify erythropoietin polypeptide variants suitable for oral administration to an individual.

Compositions containing a human erythropoietin polypeptide variant of the invention can be delivered systemically in therapeutically or prophylactically effective amounts by pulmonary administration using a variety of pulmonary delivery devices, including nebulizers, metered dose inhalers and powder inhalers. Aerosol administration of compositions of the invention can result in significant elevation of red blood cell levels. Compositions of the invention can be administered in this manner to medically treat or prevent anemia, as well as to treat or prevent other maladies related to erythropoiesis.

Naffakh et al. examined whether the secretion of erythropoietin from genetically modified cells could represent an alternative to repeated injections of the recombinant hormone for treating chronic anemias responsive to erythropoietin (Naffakh et al., Proc. Nat. Acad. Sci. 92:3194-3198 (1995)). Primary mouse skin fibroblasts were transduced with a retroviral vector in which the murine cDNA was expressed under the control of the murine phosphoglycerate kinase promoter. ‘Neo-organs’ containing the genetically modified fibroblasts embedded into collagen lattices were implanted into the peritoneal cavity of mice. Increased hematocrit and elevated serum erythropoietin concentration were observed in recipient animals over a 10-month observation period. The approach was considered applicable to the treatment of human anemias and can be used with the compositions of the invention.

Osborne et al. investigated in rats the expression and biologic effects of transplanting autologous vascular smooth muscle cells transduced with a retroviral vector encoding rat erythropoietin cDNA (Osborne et al., Proc. Nat. Acad. Sci. 92:8055-8058 (1995)). Vector-derived erythropoietin secretion caused increases in reticulocytes followed by clinically significant increases in hematocrit and hemoglobin for up to 11 weeks. There were no significant differences between control and treated animals in the number of white blood cells and platlets. Kidney and to a lesser extent liver are specific organs that synthesize Erythropoietin in response to tissue oxygenation. In the treated animals, endogenous erythropoietin mRNA was largely downregulated in kidney and absent from liver. These results indicated that vascular smooth muscle cells can be genetically modified to provide treatment of anemias due to erythropoietin deficiency and suggest that this cell type may be targeted in the treatment of other diseases requiring systemic therapeutic protein delivery. Compositions of the invention can be used in a like manner.

Similar experiments were performed by Kessler et al., who demonstrated that, following a single intramuscular administration of a recombinant adeno-associated virus (rAAV) vector containing the beta-galactosidase gene into adult mice, protein expression was detected in myofibers for at least 32 weeks Kessler et al., Proc. Nat. Acad. Sci. 93:14082-14087 (1996)). Furthermore, a single intramuscular administration of an AAV vector containing a gene for human erythropoietin into mice resulted in dose-dependent secretion of erythropoietin and corresponding increases in red blood cell production that persisted for up to 40 weeks. Primary human myocytes transduced in vitro with the AAV-Epo vector also showed dose-dependent production of erythropoietin.

The compositions and erythropoietin polypeptide variants of the invention are useful in modulating cell survival, proliferation and differentiation. For example, the compositions and polypeptides of the invention are useful in promoting erythroid precursor proliferation and can be used in vivo, ex vivo, in situ, or in vitro. For example, a composition containing a human erythropoietin polypeptide variant where the erythropoietin polypeptide variant is selected from SEQ ID NOS: 6, 14, 18, 26, 28, 40, 48, 60, 62, 74, 76, 82, 90, 92, 94, 96, 100, 102, 104, 112, 132, 134, 138, 152, 160, 166, 184, 188, -192, 206, 212, 218, 220, 222, 224, can be used to treat erythroid precursor cells derived from an individual ex vivo and then the increased proliferating cells can be administered back to the subject.

The invention provides a method for increasing erythrocytes in an individual by administering to the individual an effective amount of a composition that contains a human erythropoietin polypeptide variant containing an amino acid sequence selected from SEQ ID NOS: 2-212 and 218-224 EVEN and a pharmaceutically acceptable medium. The invention further provides a method for increasing erythrocytes in an individual by administering to the individual an effective amount of a composition that contains a human erythropoietin polypeptide variant containing an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 224 and a pharmaceutically acceptable medium.

The number of erythrocytes in an individual can be measured, for example, using a hematocrit. In one embodiment, the invention provides a method for providing increasing erythrocytes an individual by administering an effective amount of a composition containing a human erythropoietin polypeptide variant where the erythropoietin polypeptide variant is selected from SEQ ID NOS: 6, 14, 18, 26, 28, 40, 48, 60, 62, 74, 76, 82, 90, 92, 94, 96, 100, 102, 104, 112, 132, 134, 138, 152, 160, 166, 184, 188, 192, 206, 212, 218, 220, 222, 224, and SEQ ID NOS: 4, 8, 68, 70, 150, 170, 174, 198. In one embodiment, the invention provides a method for increasing erythrocytes in an individual by administering an effective amount of a composition containing a human erythropoietin polypeptide variant where the erythropoietin polypeptide variant is selected from SEQ ID NOS: 26, 70, 96, 112, 138, 152 and SEQ ID NOS: 8, 14, 28, 40, 48, 60, 62, 68, 74, 90, 94, 100, 104, 132, 150, 166, 174, 184, 206, 212.

The invention provides a method for increasing erythrocytes in an individual by administering to the individual an effective amount of a pharmaceutical composition containing a human erythropoietin polypeptide variant where the erythropoietin polypeptide variant is selected from SEQ ID NOS: 2-212 and 218-224 EVEN, and where the individual is anemic. The invention provides a method for increasing erythrocytes in an individual by administering to the individual an effective amount of a pharmaceutical composition containing a human erythropoietin polypeptide variant where the erythropoietin polypeptide variant is selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, and where the individual is anemic. For example, a polypeptide variant or composition of the invention can be administered in an amount effective to increase the hematocrit level of an anemic subject. Anemia can be caused by several factors including diet and genetic factors as well as pathologies. For example, anemia can be caused by chronic renal failure or can be induced as a side-effect of chemotherapy treatment for an individual who has cancer.

Chronic renal failure (CRF) almost invariable leads to anemia. In CRF, the main cause of renal anemia is loss of peritubular cells in the kidney which are responsible for the synthesis and secretion of erythropoietin. By the time subjects with CRF require dialysis, only about 3% have a normal hemogloblin level. In most patients the level is 6-8 g/dl. Generally hemodialysis patients tend to have more severe anemia than those receiving peritoneal dialysis.

In addition to the treatment of anemia, the invention also contemplates the treatment of iron overload disorder. A subject having an iron overload disorder is administered an effective amount of a composition of the invention to increase red blood cell production and the subject is subsequently phlebotomized to remove the excess red blood cells produced (see U.S. Pat. No. 5,013,718).

The invention provides a method for reducing an effect of a neurological condition in an individual, by administering to the individual an effective amount of a composition containing a human erythropoietin polypeptide variant having an amino acid sequence selected from SEQ ID NOS: 2-212 and 218-224 EVEN and a pharmaceutically acceptable medium. The invention further provides a method for reducing an effect of a neurological condition in an individual, by administering to the individual an effective amount of a composition containing a human erythropoietin polypeptide variant having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, and a pharmaceutically acceptable medium. For example a pharmaceutical composition of the invention can be administered prophylactically in individuals who are at risk for neurological conditions or can be used after an event such as a stroke or other neurological damage.

As described above, a neurological condition can be a pathological condition affecting neuronal or glial cells in the nervous system. Pathological conditions affecting neuronal or glial cells include ischemia, apoptosis, necrosis, oxidative or free radical damage, and excitotoxicity. For example, neurological conditions include, without limitation, cerebral and spinal ischemia, acute brain injury, spinal cord injury, retinal disease, and neurodegenerative diseases such as Alzheimer's disease, Parkinson's disease, Huntington's disease, and ALS.

In the central nervous system, neurons express erythropoietin receptor (EPOR) (see OMIM Entry 133171 for information regarding EPOR) and astrocytes produce erythropoietin. Erythropoietin has been shown to protect primary cultured neurons from NMDA receptor-mediated glutamate toxicity. Sakanaka reported in vivo evidence that erythropoietin protects neurons against ischemia-induced cell death (Sakanaka, et al., Proc. Nat. Acad. Sci. 95:4635-4640 (1998)). They presented findings suggesting that erythropoietin may exert its neuroprotective effect by reducing the nitric oxide-mediated formation of free radicals or antagonizing their toxicity. Siren et al. presented data suggesting that inhibition of neuronal apoptosis underlies short latency protective effects of erythropoietin after cerebral ischemia and other brain injuries (Siren et al. Proc. Nat. Acad. Sci. 98:4044-4049 (2001)). They suggested that evealuation of erythropoietin, a compound established as clinically safe, as neuroprotective therapy in acute brain injury is indicated.

In addition to its role as a kidney cytokine regulating hematopoiesis, erythropoietin is also produced in the brain after oxidative or nitrosative stress. The transcription factor HIF1 (see OMIM Entry 603348 for information regarding HIF1) up-regulates erythropoietin following hypoxic stimuli. Digicaylioglu and Lipton demonstrated that preconditioning with erythropoietin protects neurons in models of ischemic and degenerative damage due to excitotoxins and consequent generation of free radicals, including nitric oxide (Digicaylioglu and Lipton, Nature 412:641-647, (2001)). Activation of neuronal erythropoietin receptors prevents apoptosis induced by NMDA or nitric oxide by triggering crosstalk between the signaling pathways JAK2 and NFKB (see OMIM Entry 147796 for information regarding JAK2 and OMIM Entry 164011 for information regarding NFKB). Digicaylioglu and Lipton also demonstrated that erythropoietin receptor-mediated activation of JAK2 leads to phosphorylation of the inhibitor of NFKB (see I-kappa-B-alpha, OMIM Entry 164008), subsequent nuclear translocation of the transcription factor NFKB, and NFKB-dependent transcription of neuroprotective genes (Digicaylioglu and Lipton, supra (2001)). Transfection of cerebrocortical neurons with a dominant interfering form of JAK2 or an I-kappa-B-alpha super-repressor blocks Erythropoietin-mediated prevention of neuronal apoptosis. Thus, neuronal erythropoietin receptors activate a neuroprotective pathway that is distinct from previously well characterized JAK and NFKB functions. Moreover, this erythropoietin effect may underlie neuroprotection mediated by hypoxic-ischemic preconditioning.

Celik et al. undertook studies in a rabbit model to determine whether exogenous erythropoietin might have a protective effect in injuries to the spinal cord (Celik, et al., Proc. Nat. Acad. Sci. 99:2258-2263 (2002)). Immunocytochemistry performed using human spinal cord sections showed abundant erythropoietin receptor immunoreactivity of capillaries, especially in white matter, and motor neurons within the ventral horn. Spinal cord ischemia was produced in rabbits by occlusion of the abdominal aorta. Recombinant human erythropoietin was administered intravenously immediately after the onset of reperfusion. The authors found both an acute and a delayed beneficial action of recombinant human Erythropoietin in ischemic spinal cord injury.

Erythropoietin is upregulated by hypoxia and provides protection against apoptosis of erythroid progenitors in bone marrow and also apoptosis of brain neurons (Siren et al., supra, 2001). Grimm et al. showed in the adult mouse retina that acute hypoxia dose-dependently stimulates expression of erythropoietin, fibroblast growth factor-2 (134920), and vascular endothelial growth factor (192240) via HIF1 stablization (Grimm et al., Nature Med. 8:718-724 (2002)). Hypoxic preconditioning protects retinal morphology and function against light-induced apoptosis by interfering with caspase-1 (see OMIM Entry 147678) activation, a downstream event in the intracellular death cascade. In contrast, induction of activator protein-1 (see OMIM Entry 165160), an early event in the light-stressed retina, is not affected by hypoxia. The erythropoietin receptor, required for erythropoietin signaling, localizes to photoreceptor cells. The protective effect of hypoxic preconditioning is mimicked by systemically applied erythropoietin that crosses the blood-retina barrier and prevents apoptosis even when given therapeutically after light insult. Application of erythropoietin may, through the inhibition of apoptosis, be beneficial for the treatment of different forms of retinal disease.

In rats, Junk et al. conducted parallel studies of recombinant erythropoietin in a model of transient global retinal ischemia induced by raising intraocular pressure, which is a clinically relevant model for retinal diseases (Junk, et al., . Proc. Nat. Acad. Sci. 99:10659-10664, (2002)). They observed abundant expression of EPOR throughout the ischemic retina. Neutralization of endogenous erythropoietin with soluble EPOR exacerbated ischemic injury, which supports a role for an endogenous EPO/EPOR system in the survival and recovery of neurons after an ischemic insult. Systemic administration of recombinant erythropoietin before or immediately after retinal ischemia not only reduced histopathologic damage, but also promoted functional recovery as assessed by electroretinography. Exogenous erythropoietin also significantly diminished terminal deoxynucleotidyltransferase-mediated dUTP end labeling of neurons in the ischemic retina, implying an antiapoptotic mechanism of action. These results further established Erythropoietin as a neuroprotective agent in acute neuronal ischemia.

To evaluate whether recombinant erythropoietin improves functional outcome if administered after spinal cord injury, Gorio et al. studied 2 rodent models (Gorio et al., Proc. Nat. Acad. Sci. 99:9450-9455 (2002)). In the first model, a moderate compression was produced by applying an aneurysm clip at level T3 and administering recombinant erythropoietin immediately after release of compression; partial recovery of motor function began within 12 hours after injury and was nearly complete by 28 days. In contrast, saline-treated animals exhibited only poor recovery. In the second model, recombinant erythropoietin administration given 1 hour after injury also produced a superior recovery of function compared with saline-treated controls after a contusion at level T9. In the latter model of more severe spinal cord injury, secondary inflammation was also markedly attenuated by recombinant erythropoietin administration and associated with reduced cavitation within the spinal cord. These authors suggested that erythropoietin provides early recovery of function, especially after spinal cord compression, as well as longer-latency neuroprotective, antiinflammatory, and antiapoptotic functions.

It is understood that modifications which do not substantially affect the activity of the various embodiments of this invention are also included within the definition of the invention provided herein. Accordingly, the following examples are intended to illustrate but not limit the present invention.

### EXAMPLE I

### Generation of Erythropoietin Polypeptide Variants

A version of human erythropoietin was designed which contains optimized codons for expression in E. coli. This version of erythropoietin is called EgEpo 1.3 and is disclosed herein as SEQ ID NO:2. EgEpo1.3 contains the following changes with respect to human erythropoietin: the native leader sequence was removed and a met-asp-ile was added at the N-terminus. These three amino acids are designated as positions −3, −2, and −1 respectively so that the rest of the EgEpo1.3 polypeptide correlates with the sequence of human erythropoietin from the literature. In addition, there is an ala to thr change at amino acid 79 and the deletion of TGDR at the carboxyl terminus.

The polypeptides of the invention, SEQ ID NOS: 2-212 and 218-224 EVEN, were generated using the Egea proprietary gene assembly method as disclosed in PCT application No. WO 99/14318 and in U.S. Pat. No. 6,521,427. Methods for automated synthesis and assembly similarly can be found described, for example, in the above patent publications as well as in WO 02/081490A2, all of which are incorporated herein by reference and described further above. For EgEpo1.3, a 564 base pair sequence was assembled. A 24 bp sequence for the construct primers was added on the 5′ and 3′ ends for a construct size of 612 bp. The 24 bp primer at the 5′ end is CAGGAATTCTGTTTGGAAACTGTC (SEQ ID NO:215 ) and the primer at the 3 ′ end is ACTCTCATACCATGGAAGCTTGCA (SEQ ID NO:216). In order to assemble the gene, 13 forward 50mer oligonucleotides were generated, 12 reverse 50mer oligonucleotides were generated, and 2 terminal 25mer oligonucleotides were generated. The oligonucleotides were each purified and tested for quality.

The gene construct was designed with an upstream pelB leader sequence which directs the polypeptide to the periplasm. In addition, a stop codon was also added at the end of the mature polypeptide sequence. Assembled products were cloned, transformed, and colonies were picked for DNA sequencing. The cloning vector was pCR-T7/NT-TOPO (Invitrogen). This vector contains an upstream T7 promoter, a ribosome binding site, start codon, 6xHis tag, an Xpress epitope, and an enterokinase cleavage site. Transformation of topoisomerase ligated vector was done in BL21 host cells.

For the polypeptides listed in FIGS. 1 and 2, the following protocol was used. Targeted mutations at residues thought to be important for carbohydrate content, aggregation, and EPOR binding were generated. In particular the following amino acids were targeted: for N-linked carbohydrate changes; A30, H32, P87, W88, and P90, for aggregation: N24, N38, N83, and for EPOR binding: T44, F48, N147 and L155. The oligonucleotides encoding these mutations were designed with an “N” at a single base of the targeted codon. The designation of “N” directs the oligonucleotide sequencer to add any of the four nucleotides, A, C, T, or G, at a desired position within the oligonucleotide. Genes were synthesized, assembled, cloned, sequenced, and expressed as described above.

Variants identified to exhibit altered Epo activity were selected and modified for expression mammalian cells by substitution of the prokaryotic leader sequence with the eukaryotic leader sequence set forth in SEQ ID NO:226 and encoded by the nucleotide sequence in SEQ ID NO:225. Substitution was performed by chemical synthesis of an encoding nucleic acid and ligation into the parent sequence devoid of the corresponding leader-containing fragment. Exemplary sequences modified to contain a mammalian leader sequence functioning to direct the human erythropoietin polypeptide variant into the extracellular space include SEQ ID NOS: 218, 220, 222 and 224.

The position of the “N” within the codon was varied. The position was chosen based on two criteria: (1) a position was not selected if it could induce a stop codon when translated and (2) a position was selected based on the triplet coding for the greatest diversity of amino acid. For example, position 2 was chosen over position 3 in a codon if it translated a possible 2 amino acids rather than 1.

### EXAMPLE II

### Activities of Erythropoietin Polypeptide Variants

Erythropoietin polypeptide variant clones generated in Example I, were individually expressed in E. coli using standard procedures and tested for activity using the protocol outlined below.

The human myeloid acute leukemia cell line UT-7 (DSMZ) which is known to differentiate and proliferate in response to erythropoietin was grown under standard culture conditions of 5% CO₂ at 37° C. in 80% alpha—minimum essential media (AMEM)(Biosource) with 20% fetal bovine serum (FBS) (Biosource) and 5 ng/ml of GM-CSF (Promega). UT-7 cells were equally distributed at approximately 1000 cells/100 ml in a 96 well plate for each test and positive control well. The negative control wells contained 80% AMEM with 20% FBS. Once confluent, the cells were washed in 10% phosphate buffered saline and returned to standard culture conditions of 5% CO₂ at 37° C. in 80% AMEM with 20% FBS for 48 hr.

To each test well 5 μl of an erythropoietin polypeptide variant from an E. coli supernatant was added. In each well of a 96 well plate 10 ul of alamarBlue™ (Biosource) was added. All plates were then incubated in 5% CO₂ at 37° C. At 24, 48, 72 and 96 hours after the initial addition of alamarBlue™, spectrophotometric readings (Spectramax) were recorded at 570 nm and 600 nm. From these two values the percentage reduction of alamarBlue™ was calculated as per the manufacturers directions. The percentage reduction of alamarBlue™ is a measure of cell proliferation in response to the erythropoietin polypeptide variant, where an increase in the reduction of alamarBlue™ is correlated to an increase in cell proliferation. The same procedure was replicated using the TF1 cell line to confirm results obtained using the UT-7 cell line. Table I contains the erythroid precursor proliferation activity using the UT-7 cell line, and time of onset of activity for erythropoietin polypeptide variants of the invention.

Versions of the human erythropoietin polypeptide variants engineered for expression and secretion from mammalian cells were similarly produced and tested for cell proliferation in the alamarBlue™ assay described above. These versions correspond to SEQ ID NOS: 218, 220, 222 and 224. Briefly, the encoding nucleic acids referenced as SEQ ID NOS:217, 219, 221 and 223 were cloned into a mammalian expression vector and stably expressed in CHO K1 cells or transiently expressed in COS-7 cells following transfection. SEQ ID NOS:217, 219, 221 and 223 were modified to additionally encode a poly-His tag at the carboxyl terminus of each variant. These His tagged versions are identical to the human erythropoietin polypeptide variants shown as SEQ ID NOS:218, 220, 222 and 224 and also included the addition of six His residues to the carboxyl terminus of each amino acid sequence. Following expression in CHO cells, the His-containing versions of SEQ ID NOS :218, 220, 222 and 224 were tested in the proliferation assay described previously.

Proliferation was tested for the His-containing human erythropoietin polypeptide variants corresponding to SEQ ID NOS:218, 220, 222 and 224 either using supernatant of variant expressing cells or using purified forms of the variants. Supernatant activity was determined from both stably or transiently transfected cells for these variants and showed comparable results. Variants were purified from the stably transfected cells and the activity of these isolated forms determined.

Purification was achieved the His tag of each variant and employing a purification kit obtained from Qiagen (Valencia, Calif.). Briefly, Qiagen columns and buffers were used as supplied and procedures performed according the manufacturer's instructions. Cell culture medium was centrifuged to remove cells. Supernatants were poured off and filtered through a 0.2 μm filter to remove any cells or cellular material. Nicle charged resin (Ni—NTA Agarose beads) was added to the supernatant and mixed gently to create a slurry. The sample was incubated at 4° C. for 4 hours to allow binding of the polypeptide-His tag to the agarose. The slurry was applied to a column and washed 6 times with 6 mls of Qiagen wash buffer (50 mM NaH₂PO₄.H₂0, 300 mM NaCl and 20 mM Imidazole). Bound polypeptide was subsequently eluted from the column with 3×1 ml washes using Qiagen elution buffer (50 mM NaH₂PO₄.H₂0, 300 mM NaCl and 250 mM Imidazole). The polypeptide concentration resulting in the elutate was 0.01 ng/μl, which was used in the proliferation described above. The results of the proliferation assays for the variants shown as SEQ ID NOS: 218, 220, 222 and 224 is shown below in Table II.

TABLE I

| | SEQ | | Time taken to reach maximum |
|---|---|---|---|
| Clone | ID | % reduction of alamar | reduction of alamar blue (Hr) |
| Name | NO: | blue at 96 hr | (Speed of activity) |
| P5H10 | 82 | 53.96 | 48 |
| P5H5 | 76 | 54.54 | 48 |
| P5G9 | 74 | 64.45 | 72 |
| P5D8 | 132 | 69.02 | 72 |
| P5E7 | 48 | 68.06 | 72 |
| P1D7 | 18 | 87.94 | 48 |
| P1B10 | 6 | 103.3 | 48 |
| P5A4 | 90 | 86.77 | 72 |
| P1B1 | 14 | 73.14 | 72 |
| P1C8 | 4 | 51.44 | 48 |
| P1C4 | 26 | 54.7 | 24 |
| P5B8 | 104 | 86.58 | 72 |
| P5A10 | 96 | 63.5 | 24 |
| P5A8 | 94 | 95.25 | 72 |
| P5B3 | 100 | 86.31 | 72 |
| P5A5 | 92 | 87.5 | 48 |
| P1D5 | 28 | 65.37 | 72 |
| P5F9 | 60 | 59.27 | 72 |
| P5F10 | 62 | 57.7 | 72 |
| P6E5 | 184 | 55.89 | 72 |
| P6B11 | 166 | 63.1 | 72 |
| P6F9 | 198 | 44.34 | 48 |
| P6F3 | 192 | 67.97 | 48 |
| P6B1 | 152 | 68.63 | 24 |
| P6H11 | 212 | 85.16 | 72 |
| P5B7 | 102 | 58.8 | 48 |
| P6A1 | 138 | 67.66 | 24 |
| P5C6 | 112 | 53.53 | 24 |
| P1B12 | 8 | 44.02 | 72 |
| P6B6 | 160 | 64.82 | 48 |
| P1A4 | 40 | 73.68 | 72 |
| P5D9 | 134 | 63.49 | 48 |
| P6E11 | 188 | 65.2 | 48 |
| P5G2 | 68 | 51.68 | 72 |
| P6A10 | 150 | 51.74 | 72 |
| P6C6 | 170 | 51.13 | 48 |
| P6D2 | 174 | 46.15 | 72 |
| P5G5 | 70 | 48.54 | 24 |
| P6H1 | 206 | 59.1 | 72 |
| Tem-plate | 2 | 52.93 | 48 |

TABLE II

| | | Supernatant | | Purified | |
|---|---|---|---|---|---|
| | | Polypeptide | | polypeptide (0.01 ng/ul) | |
| | | | Time taken | | Time taken |
| | | | to reach | | to reach |
| | | % | max | % | max |
| | SEQ | reduction | reduction of | reduction | reduction |
| Clone | ID | of alamar | alamar blue | of alamar | of alamar |
| Name | NO: | blue 96 hr | (hr) | at 120 hr | blue (hr) |
| P5A4 B1 | 222 | 22.73 | 96 | 28.46 | 120 |
| P5A4_B2 | 224 | 30.17 | 96 | 18.17 | 120 |
| P1B10 B1 | 218 | 22.05 | 72 | 4.22 | 96 |
| P1B10_B2 | 220 | 24.97 | 72 | 1.25 | 96 |

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A human erythropoietin polypeptide variant comprising, a human erythropoietin amino acid sequence having an amino acid difference in two or more different Epo-modification regions and an enhanced erythropoietin activity, or a functional fragment thereof.

2. The human erythropoietin polypeptide variant of claim 1, further comprising an amino acid difference located outside an Epo-modification region.

3. The human erythropoietin polypeptide variant of claim 2, wherein said amino acid difference is located at amino acid position 146.

4. The human erythropoietin polypeptide variant of claim 1, further comprising an amino acid difference in a third Epo-modification region.

5. The human erythropoietin polypeptide variant of claim 4, further comprising an amino acid difference located outside an Epo-modification region.

6. The human erythropoietin polypeptide variant of claim 5, wherein said amino acid difference is located at amino acid position 146.

7. The human erythropoietin polypeptide variant of claim 1, wherein said enhanced erythropoietin activity comprises increased erythroid precursor proliferation.

8. The human erythropoietin polypeptide variant of claim 1, wherein said enhanced erythropoietin activity comprises increased erythroid precursor differentiation.

9. The human erythropoietin polypeptide variant of claim 1, wherein said enhanced erythropoietin activity comprises a faster time to reach a maximal level of an erythropoietin activity.

10. The human erythropoietin polypeptide variant of claim 9, wherein said enhanced erythropoietin activity comprises a faster time to reach a maximal level of erythroid precursor proliferation.

11. A human erythropdietin polypeptide variant comprising, a human erythropoietin amino acid sequence having an amino acid difference in two or more different Epo-modification regions and a moderated erythropoietin activity, or a functional fragment thereof.

12. The human erythropoietin polypeptide variant of claim 11, further comprising an amino acid difference located outside an Epo-modification region.

13. The human erythropoietin polypeptide variant of claim 12, wherein said amino acid difference is located at position 146.

14. The human erythropoietin polypeptide variant of claim 11, further comprising an amino acid difference in a third Epo-modification region.

15. The human erythropoietin polypeptide variant of claim 14, further comprising an amino acid difference located outside an Epo-modification region.

16. The human erythropoietin polypeptide variant of claim 15, wherein said amino acid difference is located at position 146.

17. The human erythropoietin polypeptide variant of claim 11, wherein said moderated erythropoietin activity comprises decreased erythroid precursor proliferation.

18. The human erythropoietin polypeptide variant of claim 11, wherein said moderated erythropoietin activity comprises decreased erythroid precursor differentiation.

19. The human erythropoietin polypeptide variant of claim 11, wherein said moderated erythropoietin activity comprises a slower time to reach a maximal level of an erythropoietin activity.

20. The human erythropoietin polypeptide variant of claim 19, wherein said moderated erythropoietin activity comprises a slower time to reach a maximal level of erythroid precursor proliferation.

21. A human erythropoietin polypeptide variant, having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, or a functional fragment thereof.

22. An isolated nucleic acid molecule, comprising a nucleic acid sequence encoding a human erythropoietin polypeptide variant having an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, or a functional fragment thereof.

23. A vector comprising the isolated nucleic acid molecule of claim 22.

24. A host cell comprising the vector of claim 23.

25. A method of producing a human erythropoietin polypeptide variant, comprising growing the host cell of claim 24 under conditions sufficient for expression of said erythropoietin polypeptide variant.

26. The method of claim 25, further comprising isolating said erythropoietin polypeptide variant from the host cell or host cell medium.

27. An antibody or functional binding fragment thereof comprising, a region that specifically binds to a human erythropoietin polypeptide variant comprising an amino acid sequence as shown in SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224.

28. A composition, comprising a human erythropoietin polypeptide variant comprising an amino acid sequence selected from SEQ ID NOS: 4, 6, 8, 14, 16, 18, 26, 28, 30, 34, 36, 38, 40, 42, 44, 48, 50, 54, 56, 60, 62, 66, 68, 70, 72, 74, 76, 82, 88, 90, 92, 94, 96, 98, 100, 102, 104, 106, 108, 112, 116, 118, 120, 122, 124, 132, 134, 138, 144, 148, 150, 152, 160, 162, 164, 166, 170, 172, 174, 184, 188, 192, 194, 196, 198, 200, 206, 210, 212, 218, 220, 222, 224, and a pharmaceutically acceptable medium.

29. A method of increasing erythrocytes in an individual, comprising administering to said individual an effective amount of a composition of claim 28.

30. The method of claim 29, wherein said individual is anemic.

31. The method of claim 29, wherein said individual has chronic renal failure.

32. The method of claim 29, wherein said individual has cancer.

33. A method for reducing an effect of a neurological condition in an individual, comprising administering to said individual an effective amount of a composition of claim 29.

34. A method of measuring erythroid precursor proliferation activity of an erythropoietin polypeptide over time, comprising:
(a) contacting an erythroid precursor cell line with said erythropoietin polypeptide and a non-toxic proliferation-sensitive dye under conditions sufficient for cell viability, and
(b) measuring a signal of said dye at various times after said contacting,
wherein changes in said signal of the dye correlate with erythroid precursor proliferation activity.

35. The method of claim 34, wherein said erythropoietin polypeptide further comprises a human erythropoietin polypeptide variant.
